(19) United States
(12) Patent Application Publication
(10) Pub. No.: US 2022/0184225 A1
(43) Pub. Date: Jun. 16, 2022

(54) FUSOGENIC PARTICLES AND RELATED METHODS FOR DELIVERING THERAPEUTIC AGENTS TO CELLS

(71) Applicant: Case Western Reserve University, Cleveland, OH (US)

(72) Inventors: John C. TILTON, Cleveland, OH (US); Zach TROYER, Cleveland, OH (US)

(21) Appl. No.: 17/605,585

(22) PCT Filed: Apr. 23, 2020
(86) PCT No.: PCT/US2020/029574

§ 371 Date: Oct. 22, 2021

Related U.S. Application Data
(60) Provisional application No. 62/837,219, filed on Apr. 23, 2019.

(51) Int. Cl. A61K 47/69; C12N 7/00; A61K 9/127; A61K 47/62; A61P 35/00

(52) U.S. Cl. CPC A61K 47/6911; C12N 7/00; C12N 2760/20222; A61K 47/62; A61P 35/00; A61K 9/1271

(57) ABSTRACT

The present disclosure relates to isolated, therapeutic agent delivery platforms and, more particularly, to engineered, fusogenic particles and related methods for targeted delivery of therapeutic agents to cells. One aspect of the present disclosure relates to an isolated, fusogenic particle including a lipid envelope associated with at least one targeting protein, and a therapeutic agent contained within the fusogenic particle. The at least one targeting protein can be a viral fusion protein or a cognate receptor of a viral fusion protein. Other aspects of the present disclosure relate to in vivo and in vitro methods for delivering therapeutic agents to cells using the fusogenic particles.

### RELATED APPLICATIONS

This application claims the benefit of U.S. Provisional Application Ser. No. 62/837,219, filed Apr. 23, 2019, which is hereby incorporated by reference in its entirety for all purposes.

### GOVERNMENT SUPPORT

This invention was made with government support under R21-AI113148 and R01-AI140847 awarded by National Institutes of Health. The government has certain rights in the invention.

### TECHNICAL FIELD

The present disclosure relates generally to isolated, therapeutic agent delivery platforms and, more particularly, to engineered, fusogenic particles and related methods for targeted delivery of agents, such as therapeutic and imaging agents to cells.

### BACKGROUND

Fusogenic particles, such as extracellular vesicles (EVs) are cell-derived membranous particles released by nearly all cells, including bacteria, archaea, fungi, plants, and metazoans. In humans and other mammals, EVs are heterogeneous and range in size from 40 nm to 5 μm. Originally thought to be a system for removal of cellular waste and maintenance of homeostasis, research into EVs over the past two decades has revealed that they play an important role in intercellular communication, particularly in the immune system and cancer.

While many effects of EVs on intercellular communication are well described, the molecular mechanisms by which EVs alter target cell behavior remain incompletely defined. There are several ways through which EVs can interact with target cells: by interaction of EV surface proteins with host cell receptors, leading to signal transduction responses; by delivery of antigens to antigen presenting cells for processing, presentation, and stimulation of immune responses; by release of contents into the extracellular space by bursting, releasing proteins that affect nearby cells; through activation of toll like receptors (TLRs) in endocytic compartments; and by delivery of proteins or nucleic acids into the target cell cytosol. However, delivery of bioactive proteins and RNAs within EVs to the target cell cytosol requires membrane fusion to evade degradation within lysosomes.

Cellular membranes do not spontaneously undergo fusion and fission, an important property which allows them to compartmentalize subcellular organelles and orchestrate intracellular vesicle transport in a tightly-regulated fashion. The most well characterized system for membrane fusion in the cell are the SNARE complexes. Another mechanism for membrane fusion is found in enveloped viruses, which are surrounded by a lipid bilayer and share size similarity with EVs. Viruses encode glycoproteins embedded in the viral membrane that bind to cognate receptor proteins on the surface of host cells and facilitate fusion and viral cargo release. Full membrane fusion is said to have occurred when the lipid bilayers of the opposing membranes are contiguous and have opened a fusion pore through which particle content mixing can occur. In contrast, in hemifusion, the outer leaflets of the opposing membranes have fused and allow partial lipid mixing, but a fusion pore has not formed and contents are not transferred. One common method in the study of membrane fusion is the use of fluorescent lipids, with can detect mixing between the lipids of separate membranes. However, this method fails to distinguish full fusion from hemifusion, and thus is not indicative of full fusion and content delivery. Indeed, a recent study investigating EV transfer of K-Ras protein to glioblastoma cells reported fluorescent lipid mixing between EV and target cell membranes, yet found no evidence of cytosolic protein delivery (Luhtala and Hunter, 2018; Failure to detect functional transfer of active K-Ras protein from extracellular vesicles into recipient cells in culture. PLoS One 13).

### SUMMARY

The present disclosure relates generally to isolated, therapeutic agent delivery platforms and, more particularly, to engineered, fusogenic particles and related methods for targeted delivery of agents, such as imaging and therapeutic agents to cells.

In one aspect, the present disclosure can include an isolated, fusogenic particle comprising a lipid envelope associated with at least one targeting protein, and a therapeutic agent contained within the fusogenic particle. The at least one targeting protein can be a viral fusion protein or a cognate receptor of a viral fusion protein.

In another aspect, the present disclosure can include a method of delivering a therapeutic agent to a cell. The method can comprise contacting the cell with an effective amount of a fusogenic particle. The fusogenic particle can comprise a lipid envelope associated with at least one targeting protein, and a therapeutic agent contained within the fusogenic particle. The at least one targeting protein can be a viral fusion protein or a cognate receptor of a viral fusion protein.

In some instances, the disclosure can include the lipid envelope can be a mono- or bi-layer lipid structure.

In one example, the lipid envelope can be a mono- or bi-layer lipid structure is an extracellular vesicle (EV).

In another example, the lipid envelope can be selected from an exosome, a microsome, an endosome, an enveloped virus, an enveloped viral-like particle, a nanosome or a vacuole.

In some instances, the viral fusion protein can be derived from a population of circulating exogenous viral fusion proteins.

In one example, the viral fusion protein can be a viral envelope glycoprotein.

In one example, the viral envelope glycoprotein can be vesicular stomatitis virus glycoprotein (VSV-G).

In some instances, the viral fusion protein can be derived from a population of endogenous viral fusion proteins.

In one example, the viral fusion protein can be derived from a human retrovirus.

In one example, the viral fusion protein can be Syncytin-1.

In some instances, the cognate receptor can be solute carrier family 1 member 5 (SLC1A5).

In some instances, the cell can be in vitro.

In some instances, the cell can be in vivo.

In one example, the cell can be a cancer cell.

In another example, the cancer cell can be located within a tumor or an organ.

In a further example, the cancer cell can express endogenous retroviral glycoproteins and the fusogenic particle includes one or more cognate receptors that bind the retroviral glycoproteins.

In another example, the cell can be a virally-infected cell.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIGS. 1a-c is a series of schematic illustrations illustrating mechanisms of EV communication (FIG. 1a), and description and validation of CCF2-AM fusion assay (FIGS. 1b-c). Of note in FIG. 1a is the middle panel depicting cytosolic delivery of RNAs and EV fusion. In FIG. 1b, a membrane-enclosed particle delivers internalized β-lactamase (Bla), which cleaves cytosolically-located esterified CCF2 in the target cell. This cleavage breaks a fluorescence resonance energy transfer (FRET) pair, switching emission spectra from 530 nm to 460 nm. In FIG. 1c, the lentivirus fusion assay is performed in Jurkat E6 cells. Loss of fusion signal in the VSV-G W72A condition indicates that full membrane fusion, and not just endocytosis, is necessary for a positive fusion signal in this assay. Bars represent average fusion levels (n=3) and error bars represent standard deviation. Statistical analysis comparing VSV-G WT and W72A conditions to PBS was an unpaired two-tailed students T test followed by Bonferroni-Dunn correction for multiple comparisons.

FIGS. 2a-c illustrate production method and characterization of β-lactamase (Bla)-incorporating extracellular vesicles (EVs). FIG. 2a is a schematic describing the method for incorporating Bla into extracellular vesicles through protein fusion. Bla is fused to the N-terminal intracellular tail of three tetraspanins: CD9, CD63, and CD81, which are enriched in EV populations. This orientation places Bla into the EV lumen. FIG. 2b is a Table listing important protein hits from data-dependent acquisition liquid-chromatography tandem-mass spectrometry (LC-MS/MS) analysis of vesicular stomatitis virus glycoprotein (VSV-G) Bla-EVs. Bla and VSV-G are both present, alongside CD9, CD63, and CD81. Syntaxin-4, a t-SNARE, and a SNARE-associated protein, STXBP3, were also detected. Other proteins listed are those commonly found in EV samples. FIG. 2c shows micrographs from transmission electron microscopy (TEM) of non-enveloped (left panel) and VSV-G (right panel) EVs. Scale bar=100 nm.

FIGS. 3a-d illustrate CCF2-AM fusion assays of extracellular vesicles (EVs) with Jurkat E6 cells. FIG. 3a shows flow cytometry plots depicting the gating strategy of the CCF2-AM fusion assay and representative fusion levels of negative controls and multiple EV conditions. The uncleaved CCF2 axis represents FRET 530 nm emission spectra from intact CCF2 dye, induced by the 408 nm violet laser. The cleaved CCF2 axis represents coumarin 460 nm emission spectra from cleaved CCF2 dye, induced by the 408 nm violet laser. FIG. 3b shows a fusion assay of non-enveloped, vesicular stomatitis virus glycoprotein (VSV-G), and VSV-G W72A mutant EVs with target Jurkat E6 calls. VSV-G addition to EVs produces high fusion signal, which is ablated by VSV-G W72A mutant. Bars represent average fusion levels (n=3) and error bars represent standard deviation. Statistical analysis comparing each EV type and concentration to PBS negative controls (represented by shaded in region on graph, no EVs range, n=3) was an unpaired two-tailed students T test followed by Bonferroni-Dunn correction for multiple comparisons. FIG. 3c shows a dose-titration fusion assay of VSV-G EVs into Jurkat E6 cells. Points represents average fusion levels (n=3) and error bars represent standard deviation. Statistical analysis comparing each EV concentration to 0 ng control (not represented on graph. n=3) was an unpaired two-tailed students T test followed by Bonferroni-Dunn correction for multiple comparisons. FIG. 3d shows VSV-G EV fusion data from FIG. 3c overlaid onto Poisson distribution predictions for EV fusion levels based on different fusion threshold requirements. Data track closely with predicted fusion values based on a fusion threshold of one EV, down to the limit of detection of the assay (˜1%).

FIG. 4 shows CCF2 fusion assays of EVs derived from multiple common cell lines into target Jurkat E6 cells. 50 μL of non-enveloped and VSV-G EVs from HeLa cervical cancer, HepG2 hepatocellular carcinoma, and U87 glioblastoma cells were added to CCF2 containing Jurkat E6 cells. Bars represent average fusion levels (n=3) and error bars represent standard deviation. Asterisks directly over error bars refer to the statistical significance of the difference between each sample and the PBS negative control, represented by the shaded-in region (No EVs range, n=3). The second set of asterisks represent the statistical significance of the comparisons between the VSV-G EVs and non-enveloped EVs from each individual cell line. Statistical analysis comparing EVs to PBS negative control or to other EVs was an unpaired two-tailed students T test, followed by Bonferroni-Dunn correction for multiple comparisons where applicable.

FIGS. 5a-b show CCF2-AM fusion assays of extracellular vesicles (EVs) containing human endogenous retrovirus (HERV) fusion protein Syncytin-1 with HEK293T target cells. FIG. 5a is a fusion assay of different EV size exclusion chromatography (SEC) purification fractions, to determine the elution pattern of Syncytin-1 EVs. 100 μL of each eluent fraction was added to HEK293T target cells. Fusogenic Syncytin-1 EVs eluted primarily in fraction 2. Bars represent average fusion, and error bars represent standard deviation. FIG. 5b (Left Panel) shows dose-titration fusion assay of Syncytin-1 and non-enveloped EVs with HEK293T target cells. Dose curves were generated and some corresponding y-values on each curve were interpolated to allow visual comparison between conditions and doses. (Right Panel) Area under the curve analysis of the dose-fusion data from the upper panel. Three individual dose curves were generated for each condition, each curve using one of the three triplicate measurements for each EV dose (x-value). The area under the curve of all 3 dose curves was measured for each condition. Bars represent the average of area under the curve values (n=3). Error bars represent standard deviation. Statistical analysis comparing different Syncytin-1 EV fractions to PBS controls or Syncytin-1 EVs to non-enveloped EVs was an unpaired two-tailed students T test, followed by Bonferroni-Dunn correction for multiple comparisons where applicable.

FIGS. 6a-b show CCF2-AM fusion assays of extracellular vesicles (EVs) from breast cancer cell lines. FIG. 6a is a fusion assay of EVs derived from MCF-7 breast cancer cells. 100 μL of EVs were added to MDA-MB-231 breast cancer target cells. MCF-7 EVs were naturally capable of fusion with MDA-MB-231 cells. Bars represent average fusion levels (n=3) and error bars represent standard deviation. FIG. 6b is a MCF-7 EV fusion assay into MDA-MB-231 cells transfected with 5 μg of SLC1A5-KanR Syncytin-1 receptor plasmid. 100 μL of EVs were added to MDA-MB-231 breast cancer target cells. MCF-7 EV fusion into MDA-MB-231 cells increased more than twofold under conditions of target cell-SLC1A5 overexpression. Bars represent average fusion levels (n=10) and error bars represent standard deviation. Statistical analyses comparing different conditions were unpaired two-tailed students T tests.

FIGS. 7a-b are a series of graphs showing SLC1A5-containing EVs fuse with Syncytin-1 expressing cancer cells. In FIG. 7a, EVs were produced by HEK293T cells with or without overexpression of the SLC1A5 receptor. EVs bearing elevated levels of SLC1A5 were capable of fusion with MDA-MB- 231 cells while EVs without overexpression of SLC1A5 were not fusogenic. In FIG. 7b, SLC1A5- EV fusion assay with multiple breast cancer cell lines. SLC1A5-EVs were capable of significant fusion with MDA-MB-231s and showed elevated fusion with MCF-7s. In contrast, no fusion was detected in T47D breast cancer cells that are not thought to express Syncytin-1. Data are represented as mean fusion levels (n=3)±SD.

FIG. 8 is a schematic illustration showing the contribution of Syncytin-1 and its cognate receptor, SLC1A5, to EV fusion-based intercellular communication in cancer. Many cancers upregulate both Syncytin-1 and SLC1A5. Cancer cells can produce EVs bearing Syncytin-1 and SLC1A5. These EVs will also package cancer-associated miRNAs associated with the producer cell. These EVs can fuse with spatially distinct tumor cells expressing surface SLC1A5 and Syncytin-1, respectively, to perform an autocrine-like form of communication. Additionally, Syncytin-1 bearing EVs can travel to the surrounding tumor microenvironment (TME), fusing with stromal cells that constitutively express SLC1A5. Fusion of these EVs into stromal cells, such as fibroblasts, delivers cancer-associated miRNAs that alter cell function. Stromal cells are then converted to tumor-supporting stromal cells, which support tumor growth, angiogenesis, and tumor invasion. In some instances, stromal cells may produce EVs containing enough SLC1A5 to fuse with Syncytin-1 expressing cancer cells. Furthermore, SLC1A5 EVs can be artificially engineered to contain cargos (therapeutic agents) designed to kill cancer cells. These artificial SLC1A5-EVs fuse specifically with Syncytin-1 expressing cancer cells. (MVB=multivesicular body).

### DETAILED DESCRIPTION

I. Definitions

Unless otherwise defined, all technical terms used herein have the same meaning as commonly understood by one of ordinary skill in the art to which the present disclosure pertains.

The methods and techniques of the present disclosure are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

The section headings are used herein for organizational purposes only and are not to be construed as in any way limiting the subject matter described.

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “effective amount” can generally refer to an amount that provides the effect, e.g., effective to reduce, eliminate, or reverse a disease or disorder, such as cancer. For example, an effective amount is an amount sufficient to effectuate a beneficial or desired clinical result. The effective amount can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations, which are routine in the art.

As used herein, the term “effective route” can generally refer to a route that provides for delivery of an agent or composition to a desired compartment, system, or location. For example, an effective route is one through which an agent or composition can be administered to provide, at the desired site of action, an amount of the agent or composition sufficient to effectuate a beneficial or desired clinical result.

As used herein, the term “pharmaceutically-acceptable carrier” can refer to any pharmaceutically-acceptable medium for the fusion particles disclosed herein. Such a medium may retain isotonicity, cell metabolism, pH, and the like. It is compatible with administration to a subject in vivo, and can be used, therefore, for cell delivery and treatment.

As used herein, the term “subject” can be used interchangeably with the term “patient” and refer to any warm-blooded organism including, but not limited to, human beings, pigs, rats, mice, dogs, goats, sheep, horses, monkeys, apes, rabbits, cattle, etc.

As used herein, the term “therapeutically effective amount” can refer to the amount of a fusogenic particle or pharmaceutical composition thereof determined to produce any therapeutic response in a subject. For example, effective therapeutic fusogenic particles or pharmaceutical compositions thereof, such as those described herein may prolong the survivability of the patient, and/or inhibit overt clinical symptoms. Treatments that are therapeutically effective within the meaning of the term can include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any number of pathological symptoms of a disease or disorder, such as cancer. In some instances, the level of treatment will be relative to the given therapeutic dose and the health status of the subject.

“Treat,” “treating,” or “treatment” are used broadly in relation to the present disclosure and each such term encompasses, among others, preventing, ameliorating, inhibiting, or curing a deficiency, dysfunction, disease, or other deleterious process, including those that interfere with and/or result from a therapy.

As used herein, the terms “cancer” and “tumor” are synonymous terms, as are the terms “cancer cell” and “tumor cell”. The term “cancer” or “tumor” can refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell.

As used herein, the term “therapeutic agent” can refer to, e.g., small molecule compounds (e.g., small molecule drugs), nucleic acids (e.g., siRNA, aptamers, short hairpin RNAs, antisense oligonucleotides, ribozymes, antagomirs, microRNA mimics or DNA) or polypeptides, e.g., antibodies (e.g., full length antibodies or antigen-binding fragments thereof, Fab fragments, or scFv fragments). In some instances, the therapeutic agent is an artificial therapeutic agent, meaning that the therapeutic agent is loaded from an exogenous source into a fusogenic particle of the present disclosure (e.g., through standard laboratory techniques). Other examples of therapeutic agents are discussed below.

As used herein, the terms “isolated” or “purified” can refer to fusogenic particles that are substantially free of cellular material or other contaminating materials (e.g., proteins) from a cell or tissue source from which the fusogenic particles are derived, or substantially free of chemical precursors or other chemicals when chemically synthesized. The language “substantially free of cellular material” includes preparations of fusogenic particles in which the fusogenic particles are separated from contaminating cells and cellular debris from which the fusogenic particles are isolated or recombinantly produced. Thus, a fusogenic particle that is substantially free of cellular material includes preparations of fusogenic particles having less than about 30%, less than about 20%, less than about 10%, or less than about 5% (by dry weight) of contaminating cells and cellular debris.

As used herein, the terms “fusogenic” or “fusogenicity” can refer to the structural and functional abilities or characteristics of a fusion particle of the present disclosure to facilitate the merger (fusion) of two lipid envelopes. This is the process through which a distinct, fusogenic particle of the present disclosure merges with a cell such that the merged fusogenic particle can release its internal cargo into the cell. In some instances, whether or not a particle is fusogenic can be determined using the nanoparticle fusion assay disclosed in Example 1 of the present disclosure.

In some instances, a nanoparticle fusion assay of the present disclosure can comprise the following steps: (1) providing a population of target cells, wherein the target cells include a selectively cleavable reporter molecule; (2) providing potentially fusogenic particles (e.g., EVs); (3) combining the target cells with the potentially fusogenic particles to form a test sample; and (4) analyzing the test sample for the presence of a signal, whereby the presence of a signal indicates that the particles are fusogenic. In one example, Step (1) can include loading the target cells with a selectively cleavable reporter molecule dye (e.g., a fluorescence resonance energy transfer (FRET) dye). Step (2) can include loading potentially fusogenic particles with a molecule (e.g., an enzyme, such as β-lactamase) that selectively cleaves the reporter molecule. The dye in the target cells will change color if it is cleaved by the enzyme (e.g., β-lactamase) delivered by the potentially fusogenic particles. For example, target cells that do not fuse with potentially fusogenic particles stay green, whereas cells that do fuse turn blue. At Step (4) detection of a signal (i.e., a color change) can be done by FRET analysis.

Advantageously, the inventors of the present disclosure developed a nanoparticle fusion assay that is sensitive enough to detect the fusion of a single particle (e.g., an extracellular vesicle) with a target cell. The fusion assay of the present disclosure is based, at least in part, on the discovery that an enzyme (e.g., β-lactamase) can be complexed to tetraspanin molecules that are incorporated into EVs. It will be appreciated, however, that the fusion assay can alternatively be performed by over-expressing β-lactamase in the target cells or by complexing β-lactamase to other proteins that are enriched in EVs. Thus, in one aspect, a highly sensitive fusion assay of the present disclosure can be performed according to Steps (1)-(4) above, but wherein Step (2) can include loading potentially fusogenic particles (e.g., EVs) with an enzyme (e.g., J3-lactamase) complexed to tetraspanin molecules.

In further instances, a fusion assay of the present disclosure can be performed such that instead of delivering β-lactamase to target cells, Cre recombinase (either in a viral-like particle or EV) is delivered, which then rearranges DNA in the target cell. For instance, target cells can activate a red fluorescent protein and luciferase if they fuse with fusogenic particles containing Cre recombinase or, alternatively, target cells can change from red-to-green if they fuse with fusogenic particles containing Cre recombinase.

As used herein, the term “viral fusion protein” can refer to a viral glycoprotein that aids in driving the fusion process between the membranes of a virus and a target cell. In some instances, a viral fusion protein can include a fusion glycoprotein from an enveloped virus belonging to the families of Orthomyxoviridae, Paramyxoviridae, Retroviridae, Filoviridae or Coronaviridae. Other examples of viral fusion proteins are listed below.

As used herein, the term “circulating exogenous viral fusion protein” can refer to viral fusion proteins that are derived from circulating exogenous viruses. Such exogenous viruses are viruses that are not part of the human genome (“exogenous” to the genome) and existing (“circulating”) in nature. Circulating exogenous viral fusion proteins can therefore include any viral envelope glycoprotein from a virus that is found in nature and is able to mediate membrane fusion with cells (e.g., human cells).

As used herein, the term “endogenous viral fusion protein” can refer to viral fusion proteins derived from viruses that infected human ancestors millions of years ago and have integrated into the human genome (“endogenous” to the genome, i.e., within it). About 8% of human DNA is viral in origin. Most of that DNA has become inactivated, but there are a number of biologically active endogenous viral proteins that include functional endogenous viral fusion proteins.

As used herein, the term “cognate receptor of a viral fusion protein” can refer to a receptor for which a targeting domain of a viral fusion protein preferentially interacts with under physiological conditions, or under in vitro conditions substantially approximating physiological conditions. The term “preferentially interacts” can be synonymous with “preferentially binding” and refer to an interaction that is statistically significantly greater in degree relative to a control. Said another way, there is a discriminatory binding of the targeting domain to its cognate receptor relative to a non-cognate receptor. Thus, a targeting domain of a viral fusion protein directs binding to a specific cognate receptor located on the plasma membrane surface of a target cell. A cognate receptor of a viral fusion protein is a protein with which the virus fusion protein interacts and drives the process of membrane fusion. For instance, the HIV glycoprotein gp160 (split into gp120/gp41) can interact with some proteins on the cell surface (such as DC-SIGN) that do not drive membrane fusion, and other proteins CD4+CCR5 or CD4+CXCR4 that do drive fusion. Cognate receptors that interact with viral fusion proteins but do not trigger the fusion mechanisms of the viral fusion protein are often called “attachment factors” and are not included as a “cognate receptor of a viral fusion protein” according to the present disclosure.

As used herein, the term “viral vector” can refer to a non-replicating, non-pathogenic virus engineered for the delivery of genetic material into cells. In viral vectors, viral genes essential for replication and virulence have been replaced with a heterogenous gene (or genes) of interest.

As used herein, the term “virus particle” or “viral particle” can refer to the extracellular form of a non-pathogenic virus, in particular a viral vector, composed of genetic material made from either DNA or RNA surrounded by a protein coat, called the capsid, and in some cases an envelope derived from portions of host cell membranes and including viral glycoproteins.

As used herein, the term “Virus Like Particle” or “VLP” can refer to self-assembling, non-replicating, non-pathogenic, genomeless particle, similar in size and conformation to intact infectious virus particle.

Also herein, where a range of numerical values is provided, it is understood that each intervening value is encompassed within the present disclosure. The upper and lower limits of these smaller ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure.

### II. Overview

The present disclosure relates generally to fusogenic particles and, more particularly, to engineered, fusogenic EVs and related methods for targeted delivery of agents, such as imaging and therapeutic agents to cells.

Fusogenic particles, such as extracellular vesicles (EVs) released from cells mediate intercellular communication with important roles in immunity and cancer. EV-mediated delivery of internal RNAs and proteins to cells requires fusion between EV- and cell membranes, but the molecular mechanisms of fusion remain unknown. The inventors of the present application developed a powerful EV-cell fusion assay and surprisingly found no evidence of EV fusion in any experiment in the absence of exogenous or endogenous viral fusion proteins, demonstrating that most EVs are not inherently fusogenic. In contrast, the inventors surprisingly found that cells expressing envelope (Env) glycoproteins from circulating viruses or human endogenous retroviruses (HERVs) produced EVs that fuse with cells. Many cancers upregulate HERV Envs and their receptors, and the inventors surprisingly found that the breast cancer cell line MCF-7 produces EVs capable of fusion in a manner regulated by the HERV-W Env Syncytin-1 and its cognate receptor SLC1A5. Advantageously, the inventors further demonstrated that SLC1A5 can also be incorporated onto EVs, enabling specific fusion with cells expressing Syncytin-1.

Based at least in part of the foregoing discoveries, the present application provides fusogenic particles as well as in vitro and in vivo methods for delivering therapeutic agents to cells using the fusogenic particles.

### III. Fusogenic Particles

One aspect of the present disclosure can include an isolated and engineered fusogenic particle comprising a lipid envelope associated with at least one targeting protein and one or more therapeutic agents contained within the fusogenic particle. The at least one targeting protein can comprise a viral fusion protein or a cognate receptor of a viral fusion protein.

In some instances, fusogenic particles or vehicles suitable for the delivery of therapeutic agents can include cytoplasm-derived structures defined by a mono- or bilayer lipid envelope. Such structures can be released by a cell spontaneously and/or after internal and/or external stimulation.

Fusogenic particles or vehicles released from a cell can be, but are not limited to: any membrane-formed vesicle enclosed by a lipid envelope naturally and/or artificially released from a cell having diameter between 1000 nm and 5 nm, between 10 nm and 1000 nm, between 20 nm and 500 nm, between 40 nm and 400 nm, or between 70 nm and 300nm, e.g., between 80 nm and 200 nm. Examples of membrane-formed vesicles released from a cell are extracellular vesicles (EVs) (e.g., micelles, liposomes, exosomes, microsomes, endosomes, nanosomes, vacuoles and multivesicular bodies), viral vectors, viral or virus particles, enveloped viruses (e.g., Herpesviridae, Coronaviridae, Hepadnaviridae, Poxviridae, Retroviridae, Paramyxoviridae, Arenaviridae, Filoviridae, Bunyaviridae, Orthomyxoviridae, Togaviridae, Flaviviridae, Hepatitis D virus), viral-like particles, and endogenous or ancestral viral-like particles.

In one example, the fusogenic particle is an isolated, engineered EV. Methods for preparing and isolating EVs are disclosed in Example 1 of the present application.

In some instances, the lipid envelope comprising the fusogenic particle is associated with at least one targeting protein. By “associated with”, it is meant that the at least one targeting protein can be part of, or interact with, the lipid envelope. For example, the at least one targeting protein can be an integral membrane protein or a peripheral membrane protein. Integral membrane proteins are a permanent part of a cell membrane and can either penetrate the membrane (transmembrane) or associate with one or the other side of a membrane (integral monotopic). Peripheral membrane proteins are transiently associated with the cell membrane, typically via a combination of hydrophobic, electrostatic, and other non-covalent interactions. The targeting protein(s) of a particular fusogenic particle can all be the same or different. For example, where a fusogenic particle includes two or more targeting proteins, the targeting proteins can be different proteins in nature (integral or peripheral membrane proteins) and/or function.

In one example, the at least one targeting protein is a viral fusion protein, such as a viral envelope glycoprotein (e.g., a viral envelope glycoprotein derived from a human retrovirus). Examples of viral fusion proteins are disclosed in PCT Publication No. WO 2020/012335.

In some instances, the viral fusion protein can be derived from a population of circulating exogenous viral fusion proteins. Non-limiting examples of viral fusion proteins derived from a population of circulating exogenous viral fusion proteins can include vesicular stomatitis virus glycoprotein (VSV-G), influenza hemagglutinin, HIV (e.g., gp16, which is processed into gp120 and gp41 subunits), Herpes simplex 1 (e.g., glycoprotein B (gB) and gH/gL), measles (e.g., hemagglutinin (H) and fusion (F) proteins), Ebola virus (e.g., glycoprotein (GP)), SARS (e.g., spike (S) protein, which is processed into S1 and S2 subunits), SARS-CoV-2b (e.g., spike (S) protein, which is processed into S1 and S2 subunits), MERS (e.g., spike (S) protein, which is processed into S1 and S2 subunits), Mokola virus (e.g., glycoprotein (G)), murine leukemia virus (e.g., surface (SU) and transmembrane (TM) proteins), Zika (e.g., prM-E), hepatitis C virus (e.g., glycoprotein El), varicella zoster virus (e.g., glycoprotein E (gE)), Epstein-Barr virus (e.g., glycoprotein B (gB) and gH/gL), and cytomegalovirus (e.g., glycoprotein B (gB) and gH/gL).

In other instances, the viral fusion protein can be derived from a population of endogenous viral fusion proteins. Non-limiting examples of viral fusion protein can be derived from a population of endogenous viral fusion proteins can include Syncytin-1 (also known as ERVWE-1) (e.g., surface (SU) and transmembrane (TM)), Syncytin-2 (e.g., surface (SU) and transmembrane (TM)), human endogenous retrovirus type K 108 (HERV-K 108) (e.g., surface (SU) and transmembrane (TM)), and EnvPbl (e.g., surface (SU) and transmembrane (TM)).

In still other instances, the at least one targeting protein can comprise a cognate receptor of a viral fusion protein. Non-limiting examples of cognate receptors of viral fusion proteins can include: low density lipoprotein receptor (LDL-R), sialic acids, cluster of differentiation 4 (CD4), chemokine receptor type 5 (CCRS), C-X-C motif chemokine receptor 4 (CXCR4), heparan sulfate, CD46, signaling lymphocyte-activation molecule (SLAM), CD46, T-cell Ig and mucin domain 1 (TIM-1), angiotensin-converting enzyme 2 (ACE2), dipeptidyl peptidase IV (DPP4), solute carrier family 1 member 5 (SLC1A5) (also known as ASCT2), solute carrier family 1 member 4 (SLC1A4) and major facilitator superfamily domain containing 2A (MFSD2a).

In one example of the present disclosure, a fusogenic particle can comprise an EV having a viral fusion protein, such as a syncytin (e.g., Syncytin-1) associated with a lipid envelope thereof, and at least one therapeutic agent contained within the EV.

Syncytins according to the present disclosure can be selected from human syncytins (e.g., HERV-W and HERV-FRD), murine syncytins (e.g., syncytin-A and syncytin-B), syncytin-Oryl, syncytin-Carl, syncytin-Ruml or their functional orthologs (Dupressoir et al., Proceedings of the National Academy of Sciences of the United States of America, 2005, 102, 725-730; Lavialle et al., Phil. Trans. R. Soc. B., 2013, 368:20120507), and functional fragments thereof comprising at least the receptor binding domain (corresponding to residues 117-144 of Syncytin-1). Syncytin-1 is encoded by the ERVW-1 gene (ENS G00000242950). By functional orthologs it is intended ortholog proteins encoded by ortholog genes and that exhibit fusogenic properties. Fusogenic properties may be assessed in fusion assays as described in Example 1 of the present disclosure and Dupressoir et al. (PNAS 2005).

Human syncytins encompasses HERV-W and HERV-FRD. Functional orthologs of these proteins can be found in Hominidae. HERV-W refers to a highly fusogenic membrane glycoprotein belonging to the family of Human Endogenous Retroviruses (HERVs). HERV-W is an envelope glycoprotein; it is also called Syncytin-1. It has the sequence indicated in Ensembl database, corresponding to Transcript ERVW-1-001, ENST00000493463. The corresponding cDNA sequence is known in the art (see, e.g., PCT Publication No. WO 2019/077150). HERV-FRD also refers to a highly fusogenic membrane glycoprotein belonging to the family of Human Endogenous Retroviruses (HERVs). HERV-FRD is an envelope glycoprotein, also called Syncytin-2. It has the sequence indicated in Ensembl database, corresponding to Transcript ERVFRD-1, ENS G00000244476. The corresponding cDNA sequence is known in the art (see, e.g., PCT Publication No. WO 2019/077150).

In another example of the present disclosure, a fusogenic particle can comprise an EV having a cognate receptor, such as SLC1A5 associated with a lipid envelope thereof, and at least one therapeutic agent contained within the EV.

In some instances, fusogenic particles of the present disclosure can include one or more therapeutic agents contained within the lipid envelope thereof. Where two or more therapeutic agents are contained within the lipid envelope, the therapeutic agents can be the same or different. The particular therapeutic agent contained within the fusogenic particle will depend upon the intended application. Where, for example, treatment of a particular cancer is sought, one or combination of the same or different therapeutic agents comprising an anti-cancer agent can be selected for incorporation into the fusogenic particle. Methods for incorporating therapeutic agents into fusion particles are known in the art (see, e.g., Villa, F. et al., Pharmaceutics, 2019 Oct 28; 11(11):557; Walker, S. et al., Theranostics, 2019; 9(26):8001-8017; Luan, X. et al., Acta Pharmacologica Sinica 38, 754-763 (2017); Anand, S. et al., Biochim Biophys Acta Proteins Proteom, 2019 Dec; 1867(12):140203; and Zdanowicz and Chroboczek, Acta Biochim Pol., 2016;63(6):469-73).

Examples of therapeutic agents are listed above. In some instances, a therapeutic agent can include an anti-cancer drug. The drug may be a natural, synthetic or recombinant molecule or agent, such as a therapeutic nucleic acid, peptide nucleic acid (PNA), protein including antibody and antibody fragment, peptide, lipid including phospholipid, lipoprotein and phospholipoprotein, sugar, small molecule, other molecule or agent, or a mixture thereof. Therapeutic nucleic acids such as therapeutic RNAs can include include antisense RNAs capable of exon skipping such as modified small nuclear RNAs (snRNAs), guide RNAs or templates for genome editing, and interfering RNAs such as shRNAs and microRNAs.

In some instances, a therapeutic agent can comprise a “gene of interest for therapy”, “gene of therapeutic interest”, “gene of interest” or “heterologous gene of interest”, which includes a therapeutic gene or a gene encoding a therapeutic protein, peptide, or RNA for treating a particular disease or disorder, such as cancer.

The therapeutic gene may be a functional version of a gene or a fragment thereof. The functional version means the wild-type version of said gene, a variant gene belonging to the same family, or a truncated version, which preserves the functionality of the encoded protein. A functional version of a gene is useful for replacement or additive gene therapy to replace a gene, which is deficient or non-functional in a patient. A fragment of a functional version of a gene is useful as recombination template for use in combination with a genome editing enzyme.

Alternatively, the gene of interest may encode a therapeutic protein including a therapeutic antibody or antibody fragment, a genome-editing enzyme or a therapeutic RNA. The gene of interest is a functional gene able to produce the encoded protein, peptide or RNA in cells of the diseased tissue, such as a tumor.

The therapeutic RNA is advantageously complementary to a target DNA or RNA sequence. For example, the therapeutic RNA is an interfering RNA such as a shRNA, a microRNA, a guide RNA (gRNA) for use in combination with a Cas enzyme or similar enzyme for genome editing, or an antisense RNA capable of exon skipping such as a modified small nuclear RNA (snRNA). The interfering RNA or microRNA may be used to regulate the expression of a target gene involved in a particular disease or disorder, such as cancer. The guide RNA in complex with a Cas enzyme or similar enzyme for genome editing may be used to modify the sequence of a target gene, in particular to correct the sequence of a mutated/deficient gene, or to modify the expression of a target gene involved in the disease or disorder. The antisense RNA capable of exon skipping is used in particular to correct a reading frame and restore expression of a deficient gene having a disrupted reading frame.

A genome-editing enzyme according to the present disclosure can include an enzyme or enzyme complex that induces a genetic modification at a target genomic locus. The genome-editing enzyme is advantageously an engineered nuclease which generates a double-strand break (DSB) in the target genomic locus, such as with no limitations, a meganuclease, zinc finger nuclease (ZFN), transcription activator-like effector-based nuclease (TALENs), Cas enzyme from clustered regularly interspaced palindromic repeats (CRISPR)-Cas system and similar enzymes. The genome-editing enzyme, in particular an engineered nuclease, is usually but not necessarily used in combination with a homologous recombination (HR) matrix or template (also named DNA donor template) which modifies the target genomic locus by double-strand break (DSB)-induced homologous recombination. In particular, the HR template may introduce a transgene of interest into the target genomic locus or repair a mutation in the target genomic locus, preferably in an abnormal or deficient gene causing a particular disease or disorder.

In another aspect, fusogenic particles of the present disclosure can be formulated as a pharmaceutical composition for administration to a subject in need thereof.

In some instances, fusogenic particles can be delivered directly or in compositions containing excipients (e.g., as pharmaceutical compositions or medicaments), as is well known in the art. In one example, fusogenic particles can be formulated as a locally administrable therapeutic composition. An effective amount of fusogenic particles can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences; and Goodman and Gilman's The Pharmacological Basis of Therapeutics, 10th Ed. (2001), Hardman, Limbird, and Gilman, eds. MacGraw Hill Intl.).

The choice of formulation for fusogenic particles for a given application will depend on a variety of factors. Prominent among these will be the species of subject, the nature of the disease or disorder being treated, its state and distribution in the subject, the nature of other therapies and agents that are being administered, the optimum route for administration, survivability via the route, the dosing regimen, and other factors that will be apparent to those skilled in the art. For instance, the choice of suitable carriers and other additives will depend on the exact route of administration and the nature of the particular dosage form.

Final formulations of an aqueous suspension of fusogenic particles will typically involve adjusting the ionic strength of the suspension to isotonicity (i.e., about 0.1 to 0.2) and to physiological pH (i.e., about pH 6.8 to 7.5). The final formulation may also contain a fluid lubricant.

In some instances, fusogenic particles can be formulated in a unit dosage injectable form, such as a solution, suspension, or emulsion. Formulations suitable for injection of fusogenic particles typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of fusogenic particles and optional additives, vehicles, and/or carrier in compositions to be administered according to the present disclosure. Typically, any additives are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

The fusogenic particles can be suspended in an appropriate excipient at a desired concentration. Suitable excipients for injection solutions are those that are biologically and physiologically compatible with the fusogenic particles and with the recipient, such as buffered saline solution or other suitable excipients. The composition for administration can be formulated, produced, and stored according to standard methods complying with proper sterility and stability.

### IV. Methods

Another aspect of the present disclosure is directed to a method of delivering a therapeutic agent to a cell. The method can comprise contacting the cell with an effective amount of a fusogenic particle. As discussed below, methods of the present disclosure can be used for therapeutic indications (e.g., treating a disease or disorder, such as cancer or an infectious disease), research, and diagnostic purposes (e.g., in vivo imaging).

In one instance, a method is provided for delivering a therapeutic agent to a cell in vitro. The method can comprise contacting the cell in vitro with an effective amount of a fusogenic particle. The method can find use, for example, in cell therapy applications. For instance, a population of cells can be contacted ex vivo with an effective amount of fusogenic particles of the present disclosure. The population of cells can be intended for administration to a subject in need thereof. For example, the population of cells can comprise progenitor or stem cells, such as mesenchymal stem cells. The fusogenic particles can be loaded with therapeutic agents appropriate for treatment of the particular disease or disorder. Additionally, the fusogenic particles can include a targeting protein with an affinity for a specific target cell or tissue. After contacting the population of cells with the fusogenic particles for a time and under conditions sufficient to permit merger between the cells and the fusion particles, the population of cells (loaded with therapeutic agents) can then be administered (e.g., formulated as a pharmaceutical composition and in an appropriate dosage, as described below), in a therapeutically effective amount, to the subject.

In another example, the method can comprise contacting the cell in vitro with an effective amount of a fusogenic particle containing one or more imaging agents. An “imaging agent” can include any molecule used to detect specific biological elements using imaging techniques. Therefore, the term encompasses molecules detectable by well known imaging techniques, such as planar scintigraphy (PS), Single Photon Emission Computed Tomography (SPECT), Positron Emission Tomography (PET), contrast-enhanced ultrasonography (CEUS), Magnetic Resonance Imaging (MRI), fluorescence spectroscopy, Computed Tomography, ultrasonography, X-ray radiography, or any combination thereof. Examples of detectable molecules are disclosed in U.S. Patent Application Publication No. 2018/0303946. The method can find use in imaging applications where, for instance, a population of cells is contacted ex vivo with an effective amount of fusogenic particles of the present disclosure. The population of cells (e.g., progenitor or stem cells) can be intended for administration to a subject in need thereof. The fusogenic particles can be loaded with one or more imaging agents appropriate for the type of imaging desired. Additionally, the fusogenic particles can include a targeting protein with an affinity for a specific target cell or tissue. After contacting the population of cells with the fusogenic particles for a time and under conditions sufficient to permit merger between the cells and the fusogenic particles, the population of cells (loaded with imaging agents) can then be administered to the subject (e.g., formulated as a pharmaceutical composition and in an appropriate dosage, as described below), whereafter the appropriate imaging technique is applied to the subject.

In another example, the method can comprise contacting the cell in vitro with an effective amount of a fusogenic particle containing one or more agents, such as an imaging agent or a therapeutic agent, for the purpose of clinical and/or basic science research. For instance, fusogenic particles can be loaded with a gene of interest and then be contacted with a cell or cell line of interest to create a cell or cell line having a desired genotype and/or phenotype. Those skilled in the art will appreciate the numerous other research applications made possible by the fusogenic particles described herein.

In another aspect, the method can comprise delivering a therapeutic agent to a cell in vivo by contacting the cell with a therapeutically effective amount of the fusogenic particles described herein. Fusogenic particles can be formulated (e.g., as a pharmaceutical composition) for administration to a subject having a disease or disorder, such as cancer or an infection to thereby treat the subject. Advantageously, administered fusogenic particles can contact one or more cells in vivo, such as diseased cells (e.g., cancer cells or cells infected with a microorganism, such as a virus) and then merge or fuse with the diseased cells so that the cargo (e.g., therapeutic agent(s)) contained within the fusogenic particles is/are released into the diseased cells. Pharmaceutical formulations, routes of administration, and dosages of fusogenic particles are discussed below.

In some instances, a subject having cancer can be treated with the fusogenic particles described herein. In one example, fusogenic particles can be loaded with any one or combination of the following therapeutic agents to treat cancer (e.g., a cancer characterized by expression over over-expression of Syncytin-1): anti-cancer drugs (e.g., doxorubicin, paclitaxel, cytarabine, mitomycin C, mitroxantrone, rapamycin, docetaxel and epirubicin); RNAs, such as cDNAs (e.g., expressing TP53, CEBPA, or FUS1), siRNAs (e.g., targeting EphA2, Grb2, PLK1, VEGF, or Bcr-Abl) and miRNAs (e.g., miR-21, miR-34a, miR-16); and proteins (e.g., targeted E3 ubiquitin ligases for oncogene degradation, caspases, and toxins, such those from shigella, diphtheria, and lethal factor). Examples of cancers characterized by expression or over-expression of Syncytin-1 can include, but are not limited to, breast cancer, endometrial cancer, prostate cancer, B-cell ALL, AML, seminomas, bladder cancer, cutaneous T cell lymphoma, and colorectal cancer.

In another example, fusogenic particles can be loaded with any one or combination of the following therapeutic agents to treat an infectious disease (e.g., a viral infection): anti-viral drugs (e.g., tamiflu, remdesivir, simeprevir, sofosbuvir, ganciclovir, tenofovir, raltegravir, darunavir, abacavir, emtricitabine); RNAs, such as cDNAs (e.g., expressing Toll like receptors, RIG-I, MDA5, cytokines (e.g., IL-2, IFN-g, TNF-a, type I and type II interferons), interferon stimulated genes (e.g., MX1, MX2, ISG15, APOBEC, IFI16, etc.), siRNAs (e.g., targeting viral genes (fusion glycoproteins, such as HIV Envelope, SARS-CoV-2 spike and influenza HA, structural proteins, such as HIV Gag subunits, SARS-CoV-2 membrane, envelope, and nucleocapsid, and influenza Ml and M2), and viral enzymes, such as reverse transcriptase, RNA-dependent RNA polymerases, integrases, proteases and helicases), and mi RNAs (e.g., hsa-mir-127-3p, hsa-mir-486-5p, hsa-mir-593-5p, mmu-mir-487-5p); and protein (e.g., targeted E3 ubiquitin ligases for viral protein degradation, caspases, toxins (shigella, diphtheria, lethal factor), and antimicrobial peptides). Advantageously, fusogenic particles with any of the foregoing therapeutic agents can be engineered to express or display the cognate receptor of a specific virus's fusion protein, thereby providing a highly specific and targeted approach for delivering therapeutic agents to virally-infected cells.

A therapeutically effective amount of fusogenic particles can be delivered to a subject having a disease or disorder via an effective route, e.g., any route that provides a suitable pharmacokinetic profile. For example, fusogenic particles can be administered intravenously (e.g., in a single bolus or infusion), as a particulate or aerosol directly to the lungs (e.g., via an inhaler), subcutaneously, intramuscularly, intra-arterially, or intraperitoneally.

Typically, fusogenic particles can be administered in an amount sufficient to provide therapeutic efficacy over the treatment time course. Therapeutic efficacy can be measured using any parameter provided herein, including improvement in any pathological feature of a particular disease or disorder, such as cancer or an infectious disease.

Doses for humans or other mammals can be determined without undue experimentation by the skilled artisan, from this disclosure, the documents cited herein, and the knowledge in the art. The dose of fusogenic particles appropriate to be used in accordance with various embodiments of the present disclosure will depend on numerous factors. The parameters that will determine optimal doses to be administered for primary and adjunctive therapy generally will include some or all of the following: the disease or disorder being treated and its stage; the species of the subject, their health, gender, age, weight, and metabolic rate; the subject's immunocompetence; other therapies being administered; and expected potential complications from the subject's history or genotype. The parameters may also include the site and/or distribution that must be targeted for the fusogenic particles to be effective, and such characteristics of the site such as accessibility to fusogenic particles. Additional parameters include co-administration with other factors (such as immunosuppressive drugs). The optimal dose in a given situation also will take into consideration the way in which the fusogenic particles are formulated, the way they are administered, and the degree to which the fusogenic particles will be localized at a target site following administration.

In some instances, fusogenic particles may be administered in an initial dose, and thereafter maintained by further administration. Fusogenic particles may be administered by one method initially, and thereafter administered by the same method or one or more different methods. The levels can be maintained by the ongoing administration of the fusogenic particles. Various embodiments administer the fusogenic particles either initially or to maintain their level in the subject or both by intravenous injection. In other instances, other forms of administration are used, dependent upon the patient's condition and other factors, discussed elsewhere herein.

fusogenic particles may be administered in many frequencies over a wide range of times. Generally, lengths of treatment will be proportional to the length of the disease process, the effectiveness of the therapies being applied, and the condition and response of the subject being treated.

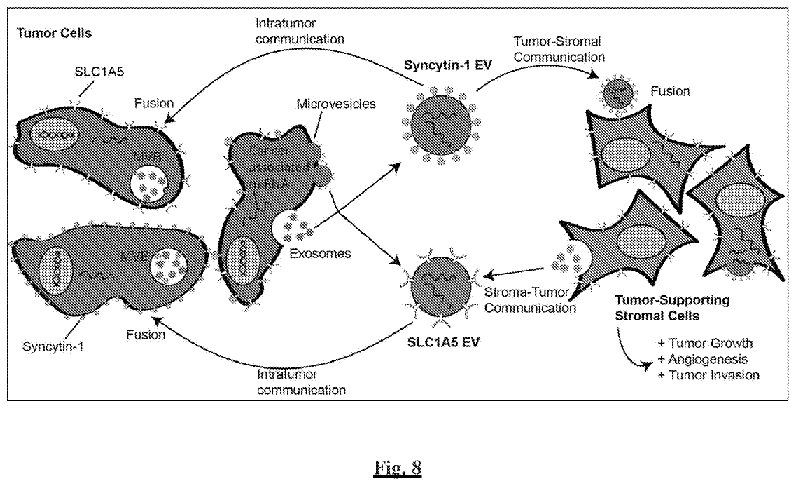

One example of the present disclosure is illustrated in FIG. 8 and includes methods for treating a cancer, such as a cancer characterized by expression or over-expression of Syncytin-1. Many cancers upregulate both Syncytin-1 and SLC1A5 and can produce EVs bearing Syncytin-1 and SLC1A5. These EVs will also package cancer-associated miRNAs associated with the producer cell. These EVs can fuse with spatially distinct tumor cells expressing surface SLC1A5 and Syncytin-1, respectively, to form an intratumoral EV communication network. Additionally, Syncytin-1 bearing EVs can travel to the surrounding tumor microenvironment (TME), fusing with stromal cells that constitutively express SLC1A5, establishing a tumor-to-stroma EV communication network. Fusion of tumor-derived EVs with stromal cells delivers cancer-associated miRNAs that alter cell function, favoring tumor growth, angiogenesis, and tumor invasion. Stromal cells may also produce EVs containing enough SLC1A5 to fuse with Syncytin-1 expressing cancer cells, establishing a stroma-to-tumor EV communication network.

Advantageously, the inventors' finding that EVs overexpressing SLC1A5 fuse with cells expressing Syncytin-1 provides an alternative anti-cancer approach; that is, fusogenic particles (e.g., EVs) bearing high levels of SLC1A5 can be manufactured to carry agents (e.g., anti-cancer drugs) that are toxic to the cancer cells. These EVs would fuse specifically with Syncytin-1 expressing cancers, killing these tumor cells while sparing bystander cells lacking aberrant HERV Env expression.

### V. EXPERIMENTAL

The following Example is for the purpose of illustration only is not intended to limit the scope of the appended claims.

### Example 1

This Example discloses the methods and results associated with fusogenic particles of the present disclosure. As discussed below, the inventors of the present application adapted a powerful virus-cell fusion assay to investigate EV fusion and surprisingly found that EVs produced by a range of cells are not inherently fusogenic. It was also unexpectedly discovered that expression of a functional viral Envelope glycoprotein (Envs) from exogenous viruses or human endogenous retroviruses (HERVs) conferred significant fusion capability to EVs.

### Methods

### Cell Lines and Maintenance

HEK293T and Jurkat E7 Cells were obtained from the ATCC repository. HeLa T4+ and U87 CD4+ CCR5+ cells were obtained from the Aids Reagent Program (ARP). HepG2 cells were a gift from Dr. Jeff Coller. MCF-7, MDA-MB-231, and T47D breast cancer cells were a gift from Dr. John Pink. Jurkat E7 Cells were grown in T25 flasks, in RPMI 1640 supplemented with 10% FBS and 1% Penicillin/Streptomycin. HEK293T cells were grown in T225 flasks, in DMEM supplemented with 10% FBS and 1% Penicillin/Streptomycin. HeLa, U87, and HepG2 cells were grown in T75 flasks, with DMEM identical to that used for HEK293Ts plus 1% non-essential amino acids. MCF-7, MDA-MB-231, and T47D cells were grown in T75 flasks, with RPMI identical to that used for E7 Jurkats plus 2mM L-Glutamine. All cells were passaged into fresh media and new flasks biweekly.Plasmids

The core plasmid for making Bla-lentiviral vectors, pNL4-34EnvAVpr-EGFP, was derived from pNL4-34Env-EGFP, obtained from the ARP. Bla-Vpr, for tagging Bla into lentiviral vectors, was generously provided by Dr. Robert Doms. Tetraspanin plasmids mEmerald-CD9-10, CD63-pEGFP C2, and mEmerald-CD81-10 were obtained from Addgene for cloning Bla-Tetraspanin constructs. Both mEmerald-CD9-10 and mEmerald-CD81-10 were gifts from Michael Davidson (Addgene plasmid # 54029/54031; http://n2t.net/addgene:54029/54031; RRID: Addgene_54029/54031). CD63-pEGFP C2 was a gift from Paul Luzio (Addgene plasmid # 62964; http://n2t.net/addgene:62964; RRID:Addgene_62964). Briefly, using Gibson assembly cloning, each tetraspanin CDS was inserted at the N terminus of Bla. VSV-G Env plasmid, also known as pMD2.G, was a gift from Didier Trono (Addgene plasmid # 12259; http://n2t.net/addgene: 12259; RRID:Addgene_12259). A different Addgene VSV-G plasmid, pCMV-VSV-G, was a gift from Bob Weinberg (Addgene plasmid # 8454; http://n2t.net/addgene:8454; RRID: Addgene_8454), and was used to clone VSV-G W72A. Using mutagenic primers to alter the two necessary base pairs, VSV-G W72A was created by Gibson assembly and confirmed by sequencing. The Syncytin-1 Env plasmid was a kind gift from Dr. Floriane Fusil at ENS Lyon. The Syncytin-1 receptor plasmid, SLC1A5, was obtained from Origene and modified for kanamycin resistance.

### Production of EVs and Lentiviral Vectors

To make LVs and EVs, 2.5×106 producer HEK293T (or HeLa, HepG2, U87, MCF-7, MDA-MB-231, T47D) cells were plated in 10 cm plates at a concentration of 2.5×105 cells/mL. HEK293Ts were transfected with polyethylenimine (PEI, Polysciences), added at a ratio of 3μg PEI/μg DNA. All other cell lines were transfected with LipofectamineTM 3000 (Invitrogen) following the manufacturer protocol for 10cm2 plates. For LVs, each plate of cells was transfected with 5μg pNL core plasmid, 3μg Bla-Vpr. and 3 μg of the desired Env plasmid. For EVs, each plate received 3μg of Env plasmid or SLC1A5-KanR, and 2μg of each Bla-Tetraspanin plasmid. EV producing cells were grown in EV-depleted media, in order to prevent contamination by bovine EVs present in FBS. Briefly, FBS was ultracentrifuged at 100,000g for 16 hours at 4° C. to pellet bovine EVs. EV-depleted FBS was then vacuum filtered through a 0.22 μM pore membrane and stored at 4° C. before being used to make EV-depleted media.

### Isolation and Purification of EVs and Lentiviral Vectors

After 3 days of LV or EV production, conditioned media was harvested from plates and clarified through a 0.45 μM filter. Conditioned media was ultracentrifuged for 2 hours at 110,000g into a 20% sucrose cushion to pellet LVs or EVs. LVs were resuspended in 250 μL DPBS−/− and stored in liquid nitrogen until further use. EVs were resuspended in 1 mL of 0.1 μM filtered DPBS−/−, then purified through sepharose CL-2B (Sigma-Aldrich) SEC columns. EV-containing eluent fractions were stored in liquid nitrogen, at 4° C., or used immediately, depending on the experiment.

### p24 ELISA and MicroBCA Assays

Titering of LVs was performed by standard p24 ELISA, using the QuickTiter™ kit (Cell Biolabs). ELISA plate was read on a ThermoScientific Multiskan FC Microplate Photometer at a wavelength of 450 nm. LV p24 concentrations were interpolated from a recombinant p24 standard curve. Titering of EVs was performed by bicinchoninic acid assay, using a MicroBCA™ Protein Assay Kit (ThermoScientific). Using the same plate reader, absorbance was measured at 570 nm, and protein concentration of EV SEC fractions determined by interpolation from a bovine serum albumin standard curve.

### CCF2-AM EV/Cell Fusion Assays

For measuring fusion of EVs with cells, target cells were plated in 96-well plates. HEK293Ts were plated at 5×105 cells/well in 50uL, while Jurkats, HeLa, HepG2, U87s, MCF-7s, MDA-MB-231s, and T47Ds were plated at 2.5×105 cells/well in 100 μL. SEC purified Bla-EVs were then added to target cells at varying volumes and concentrations. Cells were spinoculated at 1200g for 2 hours, then incubated at 37° C. for 1 hour. Target cells were then loaded with CCF2-AM dye, using the Invitrogen LiveBLAzer™ FRET-B/G Loading Kit. Cells were incubated in CO2-independent media (ThermoFisher) containing 2.5 mM probenecid at 25° C. overnight to prevent cells from removing cytosolic CCF2 dye. The following day (18 hours post probenecid incubation), target cells were washed and then fixed with 1% paraformaldehyde for flow-cytometric fusion analysis. Fusion data were collected by assessing EV-mediated cleavage of cellular CCF2 FRET dye. using a BD LSRII analytical flow cytometer. 20,000-50,000 events were collected per condition. Flow cytometry data were analyzed in FlowJo (Treestar).

Transfection of MDA-MB-231s with SLC1A5

For fusion assays into cells overexpressing SLC1A5, 2.5×106 MDA-MB-231s were plated in 10 cm plates. The following day, cells were transfected with 5 μg of SLC1A5-KanR plasmid using Lipofectamine™ 3000. The SLC1A5-KanR plasmid was Gibson assembly cloned from the original SLC1A5 plasmid from Origene, swapping out ampicillin resistance for kanamycin resistance. This was to prevent AmpR, a beta-lactamase, from interfering with the fusion assay. Three days post transfection, SLC1A5-expressing MDA-MB-231s were trypsinized and replated into 96-well plates at 2.5×105 cells/well in 100 μL of RPMI +2mM L-Glutamine The cells were then used in fusion assays as described previously.

### EV Characterization by Proteomic Analysis

For proteomic analysis of EVs, 150 μL of SEC purified EVs were lysed with 2% SDS mixed with protease inhibitor tablets (Roche Diagnostics). Detergent was removed by the previously published FASP protocol using a 10-kDa cutoff filter (Millipore), and the buffer exchanged for 8 M urea in 50 mM Tris (pH 8) to a final volume of 50 μL (Wi\grave{s}niewski et al., 2009; Universal sample preparation method for proteome analysis. Nature Methods 6, 359-362). Fifty microliters of 50mM Tris pH 8.5 was added to EV samples prior to digestion to yield a concentration of 2M Urea. The samples were reduced with 10mM dithiothreitol for 1-hour at 37° C., followed by alkylation with 25mM iodoacetaminde for 30min in the dark. The digestion was performed with 0.3 micrograms of lysyl endopeptidase on for 1 hour at 37° C. followed by the addition of 0.3 micrograms of trypsin and incubation overnight on at 37° C. Sample were analyzed by LC-MS/MS using a LTQ-Orbitrap Velos mass spectrometer (Thermo Scientific) equipped with a nanoACQUITYTM Ultra-high pressure liquid chromatography system (Waters). Mobile phases were 0.1% formic acid in water (A) and 0.1% formic acid in acetonitrile (B). Peptides were loaded onto a nanoACQUITY UPLC 2G-V/M C18 desalting trap column (180 μm×20 mm nano column, 5 μm, 100 A°) at flow rate of 0.300 μl/minute. Subsequently, peptides were resolved in a nanoACQUITY UPLC BEH300 C18 reversed phase column (75 μm×250 mm nano column, 1.7 μm, 100 AÅ; Waters) followed by a gradient elution of 1-50% of Mobile phase B in 120 minutes. A nano ES ion source at a flow rate of 300 nL/min, 1 5 kV spray voltage, and 270° C. capillary temperature was utilized to ionize peptides. Full scan MS spectra (m/z 380-1800) were acquired at a resolution of 60,000 followed by twenty data dependent MS/MS scans. MS/MS spectra were generated by collision-induced dissociation of the peptide ions (normalized collision energy=35%; activation Q=0.250; activation time=20 ms) to generate a series of b-and y-ions as major fragments. The dynamic exclusion list was confined to a maximum of 500 entries with exclusion duration of 60 seconds and mass accuracy of 10 ppm for the precursor monoisotopic mass. LC-MS/MS raw data were acquired using the Xcalibur software (Thermo Fisher Scientific, version 2.2 SP1). The LC-MS/MS raw files were processed and search using Mascot (Matrix Science London, version 2.1) and searched against the Uniprot database (550,552 sequences; 196,472,675 residues). Mascot search settings were as follow: trypsin enzyme specificity; mass accuracy window for precursor ion, 8 ppm; mass accuracy window for fragment ions, 0.8 Da; carbamidomethylation of cysteines as fixed modifications; oxidation of methionine as variable modification; and one missed cleavage. Searched files were then imported into Scaffold (Proteome Software Inc., version 4.6.2) for peptide validation and quantification by spectral count. Peptide identifications were accepted if there were >95% probability by the Peptide Prophet algorithm. Spectral counts were reported using total spectrum count for each peptide identified.

### Electron Microscopy

To image purified EVs, 2μg EVs were resuspended to 0.1 μg/ μL in a 2% paraformaldehyde solution. EVs were then prepared for EM following the previously published methods (Thery et al., 2006; Isolation and Characterization of Exosomes from Cell Culture Supernatants and Biological Fluids. Current Protocols in Cell Biology 30, 3.22.1-3.22.29). Briefly, EVs were deposited onto Formvar-carbon coated EM grids, and then washed with PBS before transfer to 1% glutaraldehyde. After water washes, EVs were contrasted in a uranyl oxalate solution (pH 7), then further contrasted and embedded in a mixture of 4% uranyl oxalate and 2% methyl cellulose. Sample grids were dried, then observed and imaged under the FEI Tecnai Spirit (T12) TEM with a Gatan US4000 4kx4k CCD at 80 kV.

### Use of Poisson Distribution to Calculate Assay Sensitivity

Poisson distribution is a discrete probability distribution used in statistics and probability theory to calculate the likelihood of a given number of events occurring in a defined space or time period. Poisson distribution has been applied in virology to model the likelihood of a cell being infected by zero, one, two, or more viruses. The probability of a cell being infected by n viruses [P(n)] can be determined using the Poisson distribution equation

$$P ( n ) = \frac{{m}^{n} \cdot {e}^{- m}}{n !}$$

where m is the multiplicity of infection (MOI, the number of viral particles in a sample divided by the number of cells), and e is Euler' s number. We reasoned that Poisson distribution could also be used to model the probability of EV fusion with a target cell in a defined space and time interval in a similar manner, with MOI in this case representing the number of EV particles divided by the number of cells. Our EV-cell fusion assay does not distinguish multiple EVs fusing with a target cell; cells are simply identified as positive or negative for CCF2 dye cleavage. However, if a single EV fusing with a target cell delivers sufficient β-lactamase to cleave CCF2, then only cells with no EV fusion [P(0)] will be detected as unfused while cells that have fused with one or more EVs [P(1)+P(2)+P(3) . . . ] will be detected as fusion-positive cells. In contrast, if a threshold (x) number of EVs is required to generate a signal, all cells with less than the threshold [P(0)+ . . . +P(x−1)] will not show evidence of fusion and dye cleavage while all cells at or above the threshold [P(x)+P(x+1)+ . . . ] will demonstrate dye cleavage. We calculated ‘effective MOIs’ such that the number of cells detected as fusion positive at thresholds of 1, 2, and 3 EVs matched the observed number of fusion positive cells (˜92.8%) obtained when 100 ng of EVs were added to target cells. Extrapolating down from these ‘effective MOIs’ with progressively lower and lower concentrations of EVs yields distinct decay curves at thresholds of 1, 2, and 3 EVs. Finally, we plotted our experimental data onto the graph of predicted curves to determine which threshold was most accurate and thus the fusion assay's sensitivity.

### Statistics

Unless otherwise noted, all Figures display mean/median fusion values (n=3) and error bars reflect standard deviation. Statistical analysis were performed by two-tailed student T test followed by Bonferroni-Dunn correction for multiple comparisons where applicable. Area under the curve analysis was performed to compare fusion dose curves of Syncytin-1 EVs to Non-Env EVs. Dose curves were generated and some corresponding y-values on each curve were interpolated to allow visual comparison between conditions and doses. For all figures, a p-value of <0.05 was considered statistically significant.

### Results

A powerful fusion assay measures nanoparticle-cell fusion and cytosolic cargo delivery

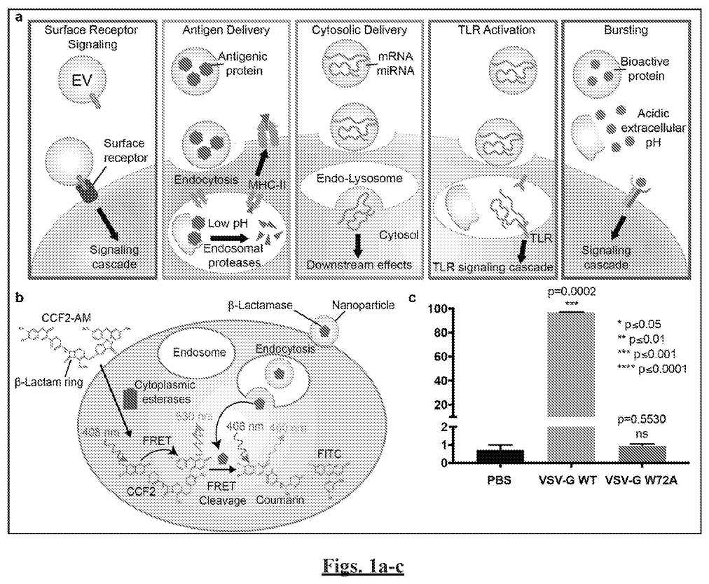

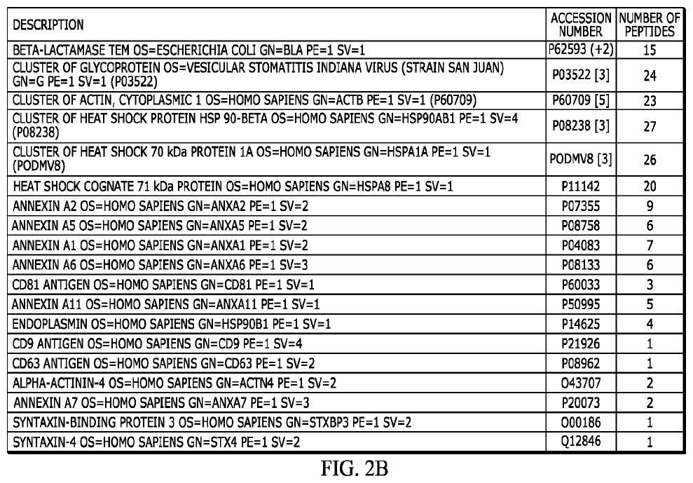

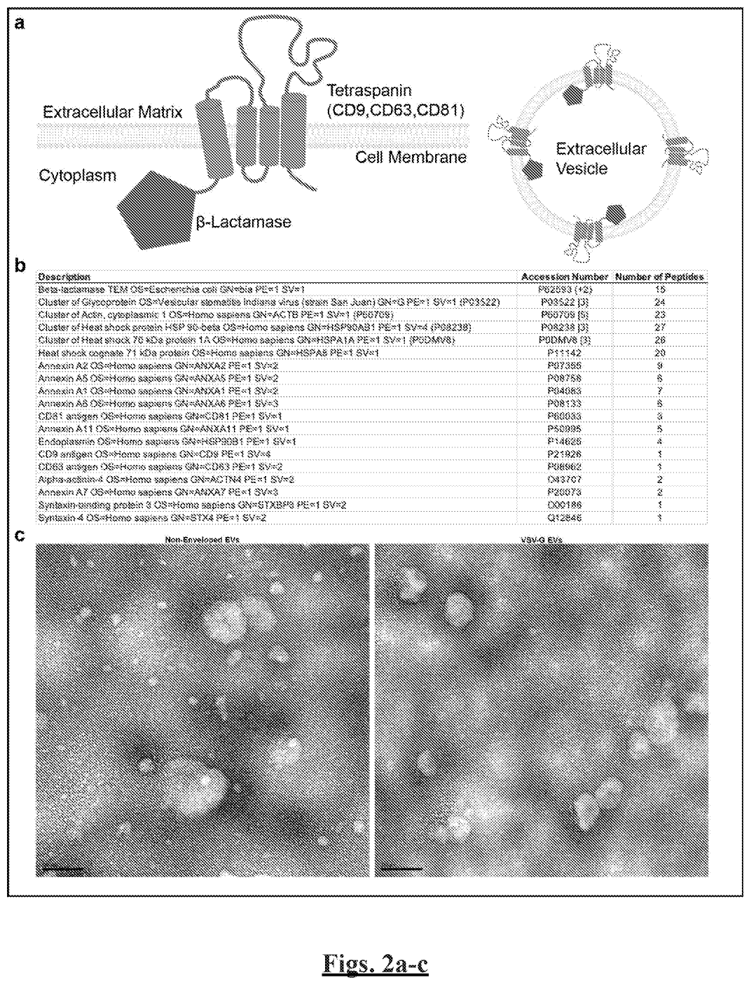

EVs are thought to interact with target cells in a variety of ways: by signaling through cellular surface receptors; through antigen delivery to antigen presenting cells; by activation of endosomal Toll-like receptors (TLRs) post uptake and degradation; by bursting in the extracellular space; and by fusion with cellular membranes to deliver internal contents to the cytosol (FIGS. 1a-b). Many studies have shown that EVs influence the function of target cells and have attributed these effects to EV-delivered cargo, particularly miRNAs. Despite strong evidence of the downstream effects of EVs, few studies have investigated EV-cell membrane fusion and none have employed assays that require full content mixing, a prerequisite for delivery of miRNAs or other internal cargos. To investigate EV fusion with cells, we utilized a far more precise fusion assay that necessitates full membrane fusion for an observable signal (FIG. 2b). In this assay, target cells are loaded with a lipophilic fluorescent dye, CCF2-AM, which upon entry into cells is esterified to CCF2 and trapped in the cytosol. This dye consists of two fluorescent moieties, coumarin and FITC, linked by a β-lactam ring. These fluorescent domains form a fluorescence resonance energy transfer (FRET) pair that is cleaved to its single components upon nanoparticle delivery of β-lactamase (Bla), resulting in a colorimetric change in the cell.

To verify that delivery of internal EV contents to the cell is required for CCF2 dye conversion, we produced Bla-carrying lentiviral particles coated with a functional, fusogenic viral Env, vesicular stomatitis virus glycoprotein (VSV-G). Concurrently, we produced identical lentiviral particles coated with VSV-G bearing a mutation (W72A) in the fusion loop that is capable of endocytic uptake but not fusion or endosomal escape (Kim et al., 2017; Mechanism of membrane fusion induced by vesicular stomatitis virus G protein. PNAS 114, E28-E36; Sun et al., 2008; Molecular Architecture of the Bipartite Fusion Loops of Vesicular Stomatitis Virus Glycoprotein G, a Class III Viral Fusion Protein. J. Biol. Chem. 283, 6418-6427). When identical amounts of these lentiviral particles were added to Jurkat E6 cells loaded with CCF2-AM, WT VSV-G particles generated a robust signal indicative of dye cleavage (p=0.0002, FIG. 2B). In contrast, Jurkats exposed to VSV-G W72A particles showed no evidence of CCF2 cleavage (p=0.5530). These data demonstrate that full membrane fusion, content mixing, and Bla escape from endocytic compartments are absolute requirements for a positive CCF2 assay signal.

Adaptation of CCF2-AM fusion assay to monitor EV-cell fusion

In order to adapt this powerful fusion assay to study EV fusion, we devised a strategy to incorporate Bla on the luminal side of EVs. Chimeric proteins were designed that consisted of Bla fused to the N-terminal intracellular tail of the three EV-enriched, membrane-bound tetraspanins: CD9, CD63, and CD81 (FIG. 2c). The orientation of Bla in the context of these chimeras was highly important: by linking Bla to the luminal side of the EV, full membrane fusion is required to expose Bla to the cytosolic CCF2 dye. We transfected HEK293T cells with these chimeric constructs and harvested EVs. Analytical mass spectrometry of purified VSV-G HEK-EVs revealed the presence of CD9, CD63, and CD81, as well as cellular proteins often associated with EVs such as actin, heat-shock proteins, and annexins (FIG. 2D and Table 1).

TABLE 1
Mass spectrometry characterization of VSV-G Bla-EVs

| | | | Alternate | Molecular | Number of |
|---|---|---|---|---|---|
| Protein # | | Accession Number | ID | Weight | Peptides |
| Non-human | | | | | |
| transfected | | | | | |
| proteins | | | | | |
| | Identified Proteins | | | | |
| 4 | Cluster of Glycoprotein | sp\|P03522\|GLYCO_VSIVA [3] | G | 57 kDa | 24 |
| | OS = Vesicular stomatitis Indiana virus | | | | |
| | (strain San Juan) | | | | |
| | OX = 11285 GN = G PE = 1 SV = 1 | | | | |
| | (sp\|P03522\|GLYCO_VSIVA) | | | | |
| 16 | Beta-lactamase TEM OS = Escherichia coli | sp\|P62593\|BLAT_ECOLX (+2) | bla | 32 kDa | 15 |
| | OX = 562 GN = bla PE = 1 SV = 1 | | | | |
| Human | | | | | |
| Proteins | | | | | |
| | Identified Proteins (185) | | | | |
| 1 | Cluster of Keratin, type II cytoskeletal 1 | sp\|P04264\|K2C1_HUMAN [3] | KRT1 | 66 kDa | 79 |
| | OS = Homo sapiens OX = 9606 | | | | |
| | GN = KRT1 PE = 1 SV = 6 | | | | |
| | (sp\|P04264\|K2C1_HUMAN) | | | | |
| 2 | Cluster of Keratin, type I cytoskeletal 16 | sp\|P08779\|K1C16_HUMAN [3] | KRT16 | 51 kDa | 54 |
| | OS = Homo sapiens OX = 9606 | | | | |
| | GN = KRT16 PE = 1 SV = 4 | | | | |
| | (sp\|P08779\|K1C16_HUMAN) | | | | |
| 3 | Cluster of Keratin, type II cytoskeletal 6A | sp\|P02538\|K2C6A_HUMAN [3] | KRT6A | 60 kDa | 43 |
| | OS = Homo sapiens OX = 9606 | | | | |
| | GN = KRT6A PE = 1 SV = 3 | | | | |
| | (sp\|P02538\|K2C6A_HUMAN) | | | | |
| 4 | Serum albumin OS = Homo sapiens | sp\|P02768\|ALBU_HUMAN | ALB | 69 kDa | 42 |
| | OX = 9606 GN = ALB PE = 1 SV = 2 | | | | |
| 5 | Keratin, type II cytoskeletal 5 | sp\|P13647\|K2C5_HUMAN | KRT5 | 62 kDa | 33 |
| | OS = Homo sapiens OX = 9606 | | | | |
| | GN = KRT5 PE = 1 SV = 3 | | | | |
| 6 | Cluster of Keratin, type II cytoskeletal 4 | sp\|P19013\|K2C4_HUMAN [4] | KRT4 | 57 kDa | 32 |
| | OS = Homo sapiens OX = 9606 | | | | |
| | GN = KRT4 PE = 1 SV = 4 | | | | |
| | (sp\|P19013\|K2C4_HUMAN) | | | | |
| 7 | Keratin, type I cytoskeletal 9 | sp\|P35527\|K1C9_HUMAN | KRT9 | 62 kDa | 29 |
| | OS = Homo sapiens OX = 9606 | | | | |
| | GN = KRT9 PE = 1 SV = 3 | | | | |
| 8 | Keratin, type I cytoskeletal 13 | sp\|P13646\|K1C13_HUMAN | KRT13 | 50 kDa | 29 |
| | OS = Homo sapiens OX = 9606 | | | | |
| | GN = KRT13 PE = 1 SV = 4 | | | | |
| 9 | Keratin, type I cytoskeletal 10 | sp\|P13645\|K1C10_HUMAN | KRT10 | 59 kDa | 27 |
| | OS = Homo sapiens OX = 9606 | | | | |
| | GN = KRT10 PE = 1 SV = 6 | | | | |
| 10 | Cluster of Heat shock protein HSP 90-beta | sp\|P08238\|HS90B_HUMAN [3] | HSP90AB1 | 83 kDa | 27 |
| | OS = Homo sapiens OX = 9606 | | | | |
| | GN = HSP90AB1 PE = 1 SV = 4 | | | | |
| | (sp\|P08238\|HS90B_HUMAN) | | | | |
| 11 | Cluster of Sodium/potassium-transporting | sp\|P05023\|AT1A1_HUMAN [2] | ATP1A1 | 113 kDa | 27 |
| | ATPase subunit alpha-1 | | | | |
| | OS = Homo sapiens OX = 9606 GN = ATP1A1 | | | | |
| | PE = 1 SV = 1 | | | | |
| | (sp\|P05023\|AT1A1_HUMAN) | | | | |
| 12 | Cluster of Heat shock 70 kDa protein 1A | sp\|P0DMV8\|HS71A_HUMAN [3] | HSPA1A | 70 kDa | 26 |
| | OS = Homo sapiens OX = 9606 | | | | |
| | GN = HSPA1A PE = 1 SV = 1 | | | | |
| | (sp\|P0DMV8\|HS71A_HUMAN) | | | | |
| 13 | Cluster of Actin, cytoplasmic 1 | sp\|P60709\|ACTB_HUMAN [5] | ACTB | 42 kDa | 23 |
| | OS = Homo sapiens OX = 9606 | | | | |
| | GN = ACTB PE = 1 SV = 1 | | | | |
| | (sp\|P60709\|ACTB_HUMAN) | | | | |
| 14 | Heat shock cognate 71 kDa protein | sp\|P11142\|HSP7C_HUMAN | HSPA8 | 71 kDa | 20 |
| | OS = Homo sapiens OX = 9606 | | | | |
| | GN = HSPA8 PE = 1 SV = 1 | | | | |
| 15 | Cluster of Hemoglobin subunit beta | sp\|P68871\|HBB_HUMAN [2] | HBB | 16 kDa | 18 |
| | OS = Homo sapiens OX = 9606 | | | | |
| | GN = HBB PE = 1 SV = 2 | | | | |
| | (sp\|P68871\|HBB_HUMAN) | | | | |
| 16 | Serotransferrin OS = Homo sapiens | sp\|P02787\|TRFE_HUMAN | TF | 77 kDa | 17 |
| | OX = 9606 GN = TF PE = 1 SV = 3 | | | | |
| 17 | Desmoplakin OS = Homo sapiens | sp\|P15924\|DESP_HUMAN | DSP | 332 kDa | 16 |
| | OX = 9606 GN = DSP PE = 1 SV = 3 | | | | |
| 18 | Keratin, type II cytoskeletal 3 | sp\|P12035\|K2C3_HUMAN | KRT3 | 64 kDa | 16 |
| | OS = Homo sapiens OX = 9606 GN = KRT3 | | | | |
| | PE = 1 SV = 3 | | | | |
| 19 | Cluster of Tubulin alpha-1B chain | sp\|P68363\|TBA1B_HUMAN [2] | TUBA1B | 50 kDa | 15 |
| | OS = Homo sapiens OX = 9606 GN = TUBA1B | | | | |
| | PE = 1 SV = 1 | | | | |
| | (sp\|P68363\|TBA1B_HUMAN) | | | | |
| 20 | Cluster of 14-3-3 protein theta | sp\|P27348\|1433T_HUMAN [4] | YWHAQ | 28 kDa | 15 |
| | OS = Homo sapiens OX = 9606 GN = YWHAQ | | | | |
| | PE = 1 SV = 1 | | | | |
| | (sp\|P27348\|1433T_HUMAN) | | | | |
| 21 | Cluster of Tubulin beta chain | sp\|P07437\|TBB5_HUMAN [3] | TUBB | 50 kDa | 14 |
| | OS = Homo sapiens OX = 9606 | | | | |
| | GN = TUBB PE = 1 SV = 2 | | | | |
| | (sp\|P07437\|TBB5_HUMAN) | | | | |
| 22 | Alpha-enolase OS = Homo sapiens | sp\|P06733\|ENOA_HUMAN | ENO1 | 47 kDa | 13 |
| | OX = 9606 GN = ENO1 PE = 1 SV = 2 | | | | |
| 23 | Cluster of Serpin B3 OS = Homo sapiens | sp\|P29508\|SPB3_HUMAN [2] | SERPINB3 | 45 kDa | 13 |
| | OX = 9606 GN = SERPINB3 PE = 1 SV = 2 | | | | |
| | (sp\|P29508\|SPB3_HUMAN) | | | | |
| 24 | Hornerin OS = Homo sapiens OX = 9606 | sp\|Q86YZ3\|HORN_HUMAN | HRNR | 282 kDa | 12 |
| | GN = HRNR PE = 1 SV = 2 | | | | |
| 25 | 4F2 cell-surface antigen heavy chain | sp\|P08195\|4F2_HUMAN | SLC3A2 | 68 kDa | 12 |
| | OS = Homo sapiens OX = 9606 GN = SLC3A2 | | | | |
| | PE = 1 SV = 3 | | | | |
| 26 | 14-3-3 protein epsilon OS = Homo sapiens | sp\|P62253\|1433E_HUMAN | YWHAE | 29 kDa | 11 |
| | OX = 9606 GN = YWHAE PE = 1 SV = 1 | | | | |
| 27 | Cluster of Ezrin OS = Homo sapiens | sp\|P15311\|EZRI_HUMAN [3] | EZR | 69 kDa | 10 |
| | OX = 9606 GN = EZR PE = 1 SV = 4 | | | | |
| | (sp\|P15311\|EZRI_HUMAN) | | | | |
| 28 | Haptoglobin OS = Homo sapiens | sp\|P00738\|HPT_HUMAN | HP | 45 kDa | 10 |
| | OX = 9606 GN = HP PE = 1 SV = 1 | | | | |
| 29 | Cluster of Cofilin-1 OS = Homo sapiens | sp\|P23528\|COF1_HUMAN [2] | CFL1 | 19 kDa | 10 |
| | OX = 9606 GN = CFL1 PE = 1 SV = 3 | | | | |
| | (sp\|P23528\|COF1_HUMAN) | | | | |
| 30 | Cluster of Guanine nucleotide-binding | sp\|P62873\|GBB1_HUMAN [2] | GNB1 | 37 kDa | 9 |
| | protein G(I)/G(S)/G(T) | | | | |
| | subunit beta-1 OS = Homo sapiens | | | | |
| | OX = 9606 GN = GNB1 PE = 1 | | | | |
| | SV = 3 (sp\|P62873\|GBB1_HUMAN) | | | | |
| 31 | Annexin A2 OS = Homo sapiens | sp\|P07355\|ANXA2_HUMAN | ANXA2 | 39 kDa | 9 |
| | OX = 9606 GN = ANXA2 PE = 1 SV = 2 | | | | |
| 32 | 14-3-3 protein zeta/delta OS = Homo sapiens | sp\|P63104\|1433Z_HUMAN | YWHAZ | 28 kDa | 9 |
| | OX = 9606 GN = YWHAZ PE = 1 SV = 1 | | | | |
| 33 | Elongation factor 1-alpha 1 OS = Homo sapiens | sp\|P68104\|EF1A1_HUMAN (+1) | EEF1A1 | 50 kDa | 9 |
| | OX = 9606 GN = EEF1A1 PE = 1 SV = 1 | | | | |
| 34 | Endoplasmic reticulum chaperone BiP | sp\|P11021\|BIP_HUMAN | HSPA5 | 72 kDa | 9 |
| | OS = Homo sapiens OX = 9606 GN = HSPA5 | | | | |
| | PE = 1 SV = 2 | | | | |
| 35 | Glyceraldehyde-3-phosphate dehydrogenase | sp\|P04406\|G3P_HUMAN | GAPDH | 36 kDa | 8 |
| | OS = Homo sapiens OX = 9606 GN = GAPDH | | | | |
| | PE = 1 SV = 3 | | | | |
| 36 | Cluster of Immunoglobulin heavy constant | sp\|P01857\|IGHG1_HUMAN [2] | IGHG1 | 36 kDa | 8 |
| | gamma 1 OS = Homo sapiens | | | | |
| | OX = 9606 GN = IGHG1 PE = 1 SV = 1 | | | | |
| | (sp\|P01857\|IGHG1_HUMAN) | | | | |
| 37 | Myristoylated alanine-rich C-kinase substrate | sp\|P29966\|MARCS_HUMAN | MARCKS | 32 kDa | 8 |
| | OS = Homo sapiens OX = 9606 GN = MARCKS | | | | |
| | PE = 1 SV = 4 | | | | |
| 38 | Keratin, type II cytoskeletal 78 OS = Homo sapiens | sp\|Q8N1N4\|K2C78_HUMAN | KRT78 | 57 kDa | 8 |
| | OX = 9606 GN = KRT78 PE = 1 SV = 2 | | | | |
| 39 | Hemoglobin subunit alpha OS = Homo sapiens | sp\|P69905\|HBA_HUMAN | HBA1 | 15 kDa | 7 |
| | OX = 9606 GN = HBA1 PE = 1 SV = 2 | | | | |
| 40 | Calnexin OS = Homo sapiens | sp\|P27824\|CALX_HUMAN | CANX | 68 kDa | 7 |
| | OX = 9606 GN = CANX PE = 1 SV = 2 | | | | |
| 41 | Cluster of Ras-related protein Rap-1b | sp\|P61224\|RAP1B_HUMAN [2] | RAP1B | 21 kDa | 7 |
| | OS = Homo sapiens OX = 9606 | | | | |
| | GN = RAP1B PE = 1 SV = 1 | | | | |
| | (sp\|P61224\|RAP1B_HUMAN) | | | | |
| 42 | Elongation factor 2 OS = Homo sapiens | sp\|P13639\|EF2_HUMAN | EEF2 | 95 kDa | 7 |
| | OX = 9606 GN = EEF2 PE = 1 SV = 4 | | | | |
| 43 | Hemopexin OS = Homo sapiens OX = 9606 | sp\|P02790\|HEMO_HUMAN | HPX | 52 kDa | 7 |
| | GN = HPX PE = 1 SV = 2 | | | | |
| 44 | Annexin A1 OS = Homo sapiens OX = 9606 | sp\|P04083\|ANXA1_HUMAN | ANXA1 | 39 kDa | 7 |
| | GN = ANXA1 PE = 1 SV = 2 | | | | |
| 45 | Sodium/potassium-transporting ATPase subunit | sp\|P54709\|AT1B3_HUMAN | ATP1B3 | 32 kDa | 7 |
| | beta-3 OS = Homo sapiens OX = 9606 | | | | |
| | GN = ATP1B3 PE = 1 SV = 1 | | | | |
| 45 | Protein-glutamine gamma-glutamyltransferase E | sp\|Q08188\|TGM3_HUMAN | TGM3 | 77 kDa | 7 |
| | OS = Homo sapiens OX = 9606 GN = TGM3 | | | | |
| | PE = 1 SV = 4 | | | | |
| 47 | Carbonic anhydrase 1 OS = Homo sapiens | sp\|P00915\|CAH1_HUMAN | CA1 | 29 kDa | 7 |
| | OX = 9606 GN = CA1 PE = 1 SV = 2 | | | | |
| 48 | Pyruvate kinase PKM OS = Homo sapiens | sp\|P14618\|KPYM_HUMAN | PKM | 58 kDa | 7 |
| | OX = 9606 GN = PKM PE = 1 SV = 4 | | | | |
| 49 | Hemoglobin subunit gamma-1 OS = Homo sapiens | sp\|P69891\|HBG1_HUMAN (+1) | HBG1 | 16 kDa | 7 |
| | OX = 9606 GN = HBG1 PE = 1 SV = 2 | | | | |
| 50 | 14-3-3 protein sigma OS = Homo sapiens | sp\|P31947\|1433S_HUMAN | SFN | 28 kDa | 7 |
| | OX = 9606 GN = SFN PE = 1 SV = 1 | | | | |
| 51 | Creatine kinase B-type OS = Homo sapiens | sp\|P12277\|KCRB_HUMAN | CKB | 43 kDa | 7 |
| | OX = 9606 GN = CKB PE = 1 SV = 1 | | | | |
| 52 | Annexin A5 OS = Homo sapiens | sp\|P08758\|ANXA5_HUMAN | ANXA5 | 36 kDa | 6 |
| | OX = 9606 GN = ANXA5 PE = 1 SV = 2 | | | | |
| 53 | Cluster of ADP-ribosylation factor 3 | sp\|P61204\|ARF3_HUMAN [3] | ARF3 | 21 kDa | 6 |
| | OS = Homo sapiens OX = 9606 GN = ARF3 | | | | |
| | PE = 1 SV = 2 (sp\|P61204\|ARF3_HUMAN) | | | | |
| 54 | Annexin A6 OS = Homo sapiens OX = 9606 | sp\|P08133\|ANXA6_HUMAN | ANXA6 | 76 kDa | 6 |
| | GN = ANXA6 PE = 1 SV = 3 | | | | |
| 55 | Fatty acid-binding protein 5 OS = Homo sapiens | sp\|Q01469\|FABP5_HUMAN | FABP5 | 15 kDa | 6 |
| | OX = 9606 GN = FABP5 PE = 1 SV = 3 | | | | |
| 56 | Fructose-bisphosphate aldolase A | sp\|P04075\|ALDOA_HUMAN | ALDOA | 39 kDa | 6 |
| | OS = Homo sapiens | | | | |
| | OX = 9606 GN = ALDOA PE = 1 SV = 2 | | | | |
| 57 | Desmoglein-1 OS = Homo sapiens | sp\|Q02413\|DSG1_HUMAN | DSG1 | 114 kDa | 6 |
| | OX = 9606 GN = DSG1 PE = 1 SV = 2 | | | | |
| 58 | Junction plakoglobin OS = Homo sapiens | sp\|P14923\|PLAK_HUMAN | JUP | 82 kDa | 6 |
| | OX = 9606 GN = JUP PE = 1 SV = 3 | | | | |
| 59 | Calmodulin-like protein 5 OS = Homo sapiens | sp\|Q9NZT1\|CALL5_HUMAN | CALML5 | 16 kDa | 6 |
| | OX = 9606 GN = CALML5 PE = 1 SV = 2 | | | | |
| 60 | Peroxiredoxin-2 OS = Homo sapiens | sp\|P32119\|PRDX2_HUMAN | PRDX2 | 22 kDa | 6 |
| | OX = 9606 GN = PRDX2 PE = 1 SV = 5 | | | | |
| 61 | Copine-3 OS = Homo sapiens OX = 9606 | sp\|O75131\|CPNE3_HUMAN | CPNE3 | 60 kDa | 6 |
| | GN = CPNE3 PE = 1 SV = 1 | | | | |
| 62 | Guanine nucleotide-binding protein | sp\|P04899\|GNAl2_HUMAN | GNAl2 | 40 kDa | 6 |
| | G(i) subunit alpha-2 OS = Homo sapiens | | | | |
| | OX = 9606 GN = GNAl2 PE = 1 SV = 3 | | | | |
| 63 | Peptidyl-prolyl cis-trans isomerase A | sp\|P62937\|PPIA_HUMAN | PPIA | 18 kDa | 5 |
| | OS = Homo sapiens OX = 9606 CN = PPIA | | | | |
| | PE = 1 SV = 2 | | | | |
| 64 | Basigin OS = Homo sapiens OX = 9606 | sp\|P35613\|BASI_HUMAN | BSG | 42 kDa | 5 |
| | GN = BSG PE = 1 SV = 2 | | | | |
| 65 | Triosephosphate isomerase OS = Homo sapiens | sp\|P60174\|TPIS_HUMAN | TPI1 | 31 kDa | 5 |
| | OX = 9606 GN = TPI1 PE = 1 SV = 3 | | | | |
| 66 | L-lactate dehydrogenase B chain | sp\|P07195\|LDHB_HUMAN | LDHB | 37 kDa | 5 |
| | OS = Homo sapiens OX = 9606 GN = LDHB | | | | |
| | PE = 1 SV = 2 | | | | |
| 67 | Protein S100-A7 OS = Homo sapiens | sp\|P31151\|S10A7_HUMAN | S100A7 | 11 kDa | 5 |
| | OX = 9606 GN = S100A7 PE = 1 SV = 4 | | | | |
| 68 | Glutathione S-transferase P OS = Homo sapiens | sp\|P09211\|GSTP1_HUMAN | GSTP1 | 23 kDa | 5 |
| | OX = 9606 GN = GSTP1 PE = 1 SV = 2 | | | | |
| 69 | Annexin A11 OS = Homo sapiens | sp\|P50995\|ANX11_HUMAN | ANXA11 | 54 kDa | 5 |
| | OX = 9606 GN = ANXA11 PE = 1 SV = 1 | | | | |
| 70 | Guanine nucleotide-binding | sp\|P08754\|GNAl3_HUMAN | GNAl3 | 41 kDa | 5 |
| | protein G(k) subunit alpha | | | | |
| | OS = Homo sapiens OX = 9606 | | | | |
| | GN = GNAl3 PE = 1 SV = 3 | | | | |
| 71 | Protein S100-A9 OS = Homo sapiens | sp\|P06702\|S10A9_HUMAN | S100A9 | 13 kDa | 4 |
| | OX = 9606 GN = S100A9 PE = 1 SV = 1 | | | | |
| 72 | Polyubiquitin-B OS = Homo sapiens | sp\|P0CG47\|UBB_HUMAN (+3) | UBB | 26 kDa | 4 |
| | OX = 9606 GN = UBB PE = 1 SV = 1 | | | | |
| 73 | Cystatin-B OS = Homo sapiens | sp\|P04080\|CYTB_HUMAN | CSTB | 11 kDa | 4 |
| | OX = 9606 GN = CSTB PE = 1 SV = 2 | | | | |
| 74 | Galectin-7 OS = Homo sapiens | sp\|P47929\|LEG7_HUMAN | LGALS7 | 15 kDa | 4 |
| | OX = 9606 GN = LGALS7 PE = 1 SV = 2 | | | | |
| 75 | Peroxiredoxin-1 OS = Homo sapiens | sp\|Q06830\|PRDX1_HUMAN | PRDX1 | 22 kDa | 4 |
| | OX = 9606 GN = PRDX1 PE = 1 SV = 1 | | | | |
| 76 | Chloride intracellular channel protein 1 | sp\|O00299\|CLIC1_HUMAN | CLIC1 | 27 kDa | 4 |
| | OS = Homo sapiens OX = 9606 GN = CLIC1 | | | | |
| | PE = 1 SV = 4 | | | | |
| 77 | Immunoglobulin kappa constant OS = Homo sapiens | sp\|P01834\|IGKC_HUMAN | IGKC | 12 kDa | 4 |
| | OX = 9606 GN = IGKC PE = 1 SV = 2 | | | | |
| 78 | Thioredoxin OS = Homo sapiens OX = 9606 | sp\|P10599\|THIO_HUMAN | TXN | 12 kDa | 4 |
| | GN = TXN PE = 1 SV = 3 | | | | |
| 79 | Neutral amno acid transporter B(0) | sp\|Q15758\|AAAT_HUMAN | SLC1A5 | 57 kDa | 4 |
| | OS = Homo sapiens OX = 9606 | | | | |
| | GN = SLC1A5 PE = 1 SV = 2 | | | | |
| 80 | Cluster of Calmodulin OS = Homo sapiens | P62158 [2] | | ? | 4 |
| | GN = CALM1 PE = 1 SV = 2 (P62158) | | | | |
| 81 | Cluster of Putative Ras-related protein Rab-1C | sp\|Q92928\|RAB1C_HUMAN [4] | RAB1C | 22 kDa | 4 |
| | OS = Homo sapiens OX = 9606 GN = RAB1C | | | | |
| | PE = 5 SV = 2 (sp\|Q92928\|RAB1C_HUMAN) | | | | |
| 82 | Endoplasmin OS = Homo sapiens | sp\|P14625\|ENPL_HUMAN | HSP90B1 | 92 kDa | 4 |
| | OX = 9606 GN = HSP90B1 PE = 1 SV = 1 | | | | |
| 83 | Hsc70-interacting protein OS = Homo sapiens | sp\|P50502\|F10A1_HUMAN | ST13 | 41 kDa | 4 |
| | OX = 9606 GN = ST13 PE = 1 SV = 2 | | | | |
| 84 | Desmocollin-1 OS = Homo sapiens OX = 9606 | sp\|Q08554\|DSC1_HUMAN | DSC1 | 100 kDa | 4 |
| | GN = DSC1 PE = 1 SV = 2 | | | | |
| 85 | Prostaglandin E synthase 3 OS = Homo sapiens | sp\|Q15185\|TEBP_HUMAN | PTGES3 | 19 kDa | 4 |
| | OX = 9606 GN = PTGES3 PE = 1 SV = 1 | | | | |
| 86 | L-lactate dehydrogenase A chain OS = Homo sapiens | sp\|P00338\|LDHA_HUMAN | LDHA | 37 kDa | 4 |
| | OX = 9606 GN = LDHA PE = 1 SV = 2 | | | | |
| 87 | Transferrin receptor protein 1 OS = Homo sapiens | sp\|P02786\|TFR1_HUMAN | TFRC | 85 kDa | 4 |
| | OX = 9606 GN = TFRC PE = 1 SV = 2 | | | | |
| 88 | Caspase-14 OS = Homo sapiens OX = 9606 | sp\|P31944\|CASPE_HUMAN | CASP14 | 28 kDa | 4 |
| | GN = CASP14 PE = 1 SV = 2 | | | | |
| 89 | Rab GDP dissociation inhibitor beta | sp\|P50395\|GDIB_HUMAN | GDI2 | 51 kDa | 4 |
| | OS = Homo sapiens OX = 9606 GN = GDI2 | | | | |
| | PE = 1 SV = 2 | | | | |
| 90 | Ras-related protein Rab-5C OS = Homo sapiens | sp\|P51148\|RAB5C_HUMAN | RAB5C | 23 kDa | 4 |
| | OX = 9606 GN = RAB5C PE = 1 SV = 2 | | | | |
| 91 | Cell division control protein 42 homolog | sp\|P60953\|CDC42_HUMAN | CDC42 | 21 kDa | 4 |
| | OS = Homo sapiens OX = 9606 GN = CDC42 | | | | |
| | PE = 1 SV = 2 | | | | |
| 92 | 60S acidic ribosomal protein P0 OS = Homo sapiens | sp\|P05388\|RLA0_HUMAN | RPLP0 | 34 kDa | 4 |
| | OX = 9606 GN = RPLP0 PE = 1 SV = 1 | | | | |
| 93 | Protein S100-A8 OS = Homo sapiens | sp\|P05109\|S10A8_HUMAN | S100A8 | 11 kDa | 3 |
| | OX = 9606 GN = S100A8 PE = 1 SV = 1 | | | | |
| 94 | CD81 antigen OS = Homo sapiens | sp\|P60033\|CD81_HUMAN | CD81 | 26 kDa | 3 |
| | OX = 9606 GN = CD81 PE = 1 SV = 1 | | | | |
| 95 | Cystatin-A OS = Homo sapiens | sp\|P01040\|CYTA_HUMAN | CSTA | 11 kDa | 3 |
| | OX = 9606 GN = CSTA PE = 1 SV = 1 | | | | |
| 96 | MARCKS-related protein OS = Homo sapiens | sp\|P49006\|MRP_HUMAN | MARCKSL1 | 20 kDa | 3 |
| | OX = 9606 GN = MARCKSL1 PE = 1 SV = 2 | | | | |
| 97 | Programmed cell death 6-interacting protein | sp\|Q8WUM4\|PDC6I_HUMAN | PDCD6IP | 96 kDa | 3 |
| | OS = Homo sapiens OX = 9606 GN = PDCD6IP | | | | |
| | PE = 1 SV = 1 | | | | |
| 98 | Heat shock protein beta = 1 OS = Homo sapiens | sp\|P04792\|HSPB1_HUMAN | HSPB1 | 23 kDa | 3 |
| | OX = 9606 GN = HSPB1 PE = 1 SV = 2 | | | | |
| 99 | Ig lambda-2 chain C regions OS = Homo sapiens | P0CG05 (+1) | | ? | 3 |
| | GN = IGLC2 PE = 1 SV = 1 | | | | |
| 100 | Phosphoglycerate kinase 1 OS = Homo sapiens | sp\|P00553\|PGK1_HUMAN | PGK1 | 45 kDa | 3 |
| | OX = 9606 GN = PGK1 PE = 1 SV = 3 | | | | |
| 101 | Filaggrin OS = Homo sapiens OX = 9606 | sp\|P20930\|FILA_HUMAN | FLG | 435 kDa | 3 |
| | GN = FLG PE = 1 SV = 3 | | | | |
| 102 | Programmed cell death protein 6 OS = Homo sapiens | sp\|O75340\|PDCD6_HUMAN | PDCD6 | 22 kDa | 3 |
| | OX = 9606 GN = PDCD6 PE = 1 SV = 1 | | | | |
| 103 | Superoxide dismutase [Cu—Zn] OS = Homo sapiens | sp\|P00441\|SODC_HUMAN | SOD1 | 16 kDa | 3 |
| | OX = 9606 GN = SOD1 PE = 1 SV = 2 | | | | |
| 104 | Ras-related protein Rab-11A OS = Homo sapiens | sp\|P62491\|RB11A_HUMAN (+1) | RA811A | 24 kDa | 3 |
| | OX = 9606 GN = RAB11A PE = 1 SV = 3 | | | | |
| 105 | Cornulin OS = Homo sapiens OX = 9606 | sp\|Q9UBG3\|CRNN_HUMAN | CRNN | 54 kDa | 3 |
| | GN = CRNN PE = 1 SV = 1 | | | | |
| 106 | 60S acidic ribosomal protein P2 OS = Homo sapiens | sp\|P05387\|RLA2_HUMAN | RPLP2 | 12 kDa | 3 |
| | OX = 9606 GN = RPLP2 PE = 1 SV = 1 | | | | |
| 107 | Protein S100-A14 OS = Homo sapiens | sp\|Q9HCY8\|S10AE_HUMAN | S100A14 | 12 kDa | 3 |
| | OX = 9606 GN = S100A14 PE = 1 SV = 1 | | | | |
| 108 | Alpha-1-antitrypsin OS = Homo sapiens | sp\|P01009\|A1AT_HUMAN | SERPINA1 | 47 kDa | 3 |
| | OX = 9606 GN = SERPINA1 PE = 1 SV = 3 | | | | |
| 109 | Transthyretin OS = Homo sapiens OX = 9606 | sp\|P02766\|TTHY_HUMAN | TTR | 16 kDa | 3 |
| | GN = TTR PE = 1 SV = 1 | | | | |
| 110 | Clathrin heavy chain 1 OS = Homo sapiens | sp\|Q00610\|CLH1_HUMAN | CLTC | 192 kDa | 3 |
| | OX = 9606 GN = CLTC PE = 1 SV = 5 | | | | |
| 111 | Band 4.1-like protein 2 OS = Homo sapiens | sp\|O4349\|E41L2_HUMAN | EPB41L2 | 113 kDa | 3 |
| | OX = 9606 GN = EPB41L2 PE = 1 SV = 1 | | | | |
| 112 | Plastin-3 OS = Homo sapiens OX = 9606 | sp\|P13797\|PLST_HUMAN | PLS3 | 71 kDa | 3 |
| | GN = PLS3 PE = 1 SV = 4 | | | | |
| 113 | DnaJ homolog subfamily A member 1 | sp\|P31689\|DNJA1_HUMAN | DNAJA1 | 45 kDa | 3 |
| | OS = Homo sapiens OX = 9606 GN = DNAJA1 | | | | |
| | PE = 1 SV = 2 | | | | |
| 114 | Prolactin-inducible protein OS = Homo sapiens | sp\|P12273\|P1P_HUMAN | PIP | 17 kDa | 3 |
| | OX = 9606 GN = PIP PE = 1 SV = 1 | | | | |
| 115 | Suprabasin OS = Homo sapiens OX = 9606 | sp\|Q6UWP8\|SBSN_HUMAN | SBSN | 61 kDa | 3 |
| | GN = SBSN PE = 1 SV = 2 | | | | |
| 116 | Monocarboxylate transporter 1 OS = Homo sapiens | sp\|P53985\|MOT1_HUMAN | SLC16A1 | 54 kDa | 3 |
| | OX = 9606 GN = SLC16A1 PE = 1 SV = 3 | | | | |
| 117 | Guanine nucleotide-binding protein | sp\|P63092\|GNAS2_HUMAN (+1) | GNAS | 46 kDa | 3 |
| | G(s) subunit alpha isoforms short | | | | |
| | OS = Homo sapiens OX = 9606 CN = GNAS | | | | |
| | PE = 1 SV = 1 | | | | |
| 118 | Vimentin OS = Homo sapiens OX = 9606 | sp\|P08670\|VIME_HUMAN | VIM | 54 kDa | 3 |
| | GN = VIM PE = 1 SV = 4 | | | | |
| 119 | 60S ribosomal protein L7 OS = Homo sapiens | sp\|P18124\|RL7_HUMAN | RPL7 | 29 kDa | 2 |
| | OX = 9606 GN = RPL7 PE = 1 SV = 1 | | | | |
| 120 | Alpha-2-HS-glycoprotein OS = Homo sapiens | sp\|P02765\|FETUA_HUMAN | AHSG | 39 kDa | 2 |
| | OX = 9606 GN = AHSG PE = 1 SV = 1 | | | | |
| 121 | Fibrinogen gamma chain OS = Homo sapiens | sp\|P02679\|FIBG_HUMAN | FGG | 52 kDa | 2 |
| | OX = 9606 GN = FGG PE = 1 SV = 3 | | | | |
| 122 | Dermcidin OS = Homo sapiens OX = 9606 | sp\|P81605\|DCD_HUMAN | DCD | 11 kDa | 2 |
| | GN = DCD PE = 1 SV = 2 | | | | |
| 123 | Membrane-associated progesterone receptor | sp\|O00264\|PGRC1_HUMAN | PGRMC1 | 22 kDa | 2 |
| | component 1 OS = Homo sapiens OX = 9606 | | | | |
| | GN = PGRMC1 PE = 1 SV = 3 | | | | |
| 124 | Profilin-1 OS = Homo sapiens OX = 9606 | sp\|P07737\|PROF1_HUMAN | PFN1 | 15 kDa | 2 |
| | GN = PFN1 PE = 1 SV = 2 | | | | |
| 125 | Filaggrin-2 OS = Homo sapiens OX = 9606 | sp\|Q5D862\|FILA2_HUMAN | FLG2 | 248 kDa | 2 |
| | GN = FLG2 PE = 1 SV = 1 | | | | |
| 126 | Protein S100-A11 OS = Homo sapiens | sp\|P31949\|S10AB_HUMAN | S100A11 | 12 kDa | 2 |
| | OX = 9606 GN = S100A11 PE = 1 SV = 2 | | | | |
| 127 | Zinc-alpha-2-glycoprotein OS = Homo sapiens | sp\|P25311\|ZA2G_HUMAN | AZGP1 | 34 kDa | 2 |
| | OX = 9606 GN = AZGP1 PE = 1 SV = 2 | | | | |
| 128 | Calreticulin OS = Homo sapiens | sp\|P27797\|CALR_HUMAN | CALR | 48 kDa | 2 |
| | OX = 9606 GN = CALR PE = 1 SV = 1 | | | | |
| 129 | 2′,3′-cyclic-nucleotide 3′-phosphodiesterase | sp\|P09543\|CN37_HUMAN | CNP | 48 kDa | 2 |
| | OS = Homo sapiens OX = 9606 GN = CNP | | | | |
| | PE = 1 SV = 2 | | | | |
| 130 | Insulin receptor substrate 4 OS = Homo sapiens | sp\|O14654\|IRS4_HUMAN | IRS4 | 134 kDa | 2 |
| | OX = 9606 GN = IRS4 PE = 1 SV = 1 | | | | |
| 131 | Eukaryotic translation initiation factor 5A-1 | sp\|P63241\|IF5A1_HUMAN | EIF5A | 17 kDa | 2 |
| | OS = Homo sapiens OX = 9606 GN = EIF5A | | | | |
| | PE = 1 SV = 2 | | | | |
| 132 | Histone H2A type 1-B/E OS = Homo sapiens | sp\|P04908\|H2A1B_HUMAN (+9) | HIST1H2AB | 14 kDa | 2 |
| | OX = 9606 GN = HIST1H2AB PE = 1 SV = 2 | | | | |
| 133 | EH domain-containing protein 4 OS = Homo sapiens | sp\|Q9H223\|EHD4_HUMAN | EHD4 | 61 kDa | 2 |
| | OX = 9606 GN = EHD4 PE = 1 SV = 1 | | | | |
| 134 | Syntenin-1 OS = Homo sapiens OX = 9606 | sp\|O00560\|SDCB1_HUMAN | SDCBP | 32 kDa | 2 |
| | GN = SDCBP PE = 1 SV = 1 | | | | |
| 135 | Nucleophosmin OS = Homo sapiens | sp\|P06748\|NPM_HUMAN | NPM1 | 33 kDa | 2 |
| | OX = 9606 GN = NPM1 PE = 1 SV = 2 | | | | |
| 136 | CD99 antigen OS = Homo sapiens OX = 9606 | sp\|P14209\|CD99_HUMAN | CD99 | 19 kDa | 2 |
| | GN = CD99 PE = 1 SV = 1 | | | | |
| 137 | Alpha-actinin-4 OS = Homo sapiens | sp\|O43707\|ACTN4_HUMAN | ACTN4 | 105 kDa | 2 |
| | OX = 9606 GN = ACTN4 PE = 1 SV = 2 | | | | |
| 138 | Heterogeneous nuclear ribonucleoprotein H | sp\|P31943\|HNRH1_HUMAN | HNRNPH1 | 49 kDa | 2 |
| | OS = Homo sapiens OX = 9606 GN = HNRMPH1 | | | | |
| | PE = 1 SV = 4 | | | | |
| 139 | Ras-related protein Ral-A OS = Homo sapiens | sp\|P11233\|RALA_HUMAN | RALA | 24 kDa | 2 |
| | OX = 9606 GN = RALA PE = 1 SV = 1 | | | | |
| 140 | Arginase-1 OS = Homo sapiens OX = 9606 | sp\|P05089\|ARGI1_HUMAN | ARG1 | 35 kDa | 2 |
| | GN = ARG1 PE = 1 SV = 2 | | | | |
| 141 | EH domain-containing protein 1 OS = Homo sapiens | sp\|Q9H4M9\|EHD1_HUMAN | EHD1 | 61 kDa | 2 |
| | OX = 9606 GN = EHD1 PE = 1 SV = 2 | | | | |
| 142 | Protein S100-A6 OS = Homo sapiens | sp\|P06703\|S10A6_HUMAN | S100A6 | 10 kDa | 2 |
| | OX = 9606 GN = S100A6 PE = 1 SV = 1 | | | | |
| 143 | Dolichyl-diphosphooligosaccharide-protein | sp\|P04844\|RPN2_HUMAN | RPN2 | 69 kDa | 2 |
| | glycosyltransferase subunit 2 | | | | |
| | OS = Homo sapiens OX = 9606 GN = RPN2 | | | | |
| | PE = 1 SV = 3 | | | | |
| 144 | Histone H4 OS = Homo sapiens | sp\|P62805\|H4_HUMAN | HIST1H4A | 11 kDa | 2 |
| | OX = 9606 GN = HIST1H4A PE = 1 SV = 2 | | | | |
| 145 | Protein disulfide-isomerase OS = Homo sapiens | sp\|P07237\|PDIA1_HUMAN | P4HB | 57 kDa | 2 |
| | OX = 9606 GN = P4HB PE = 1 SV = 3 | | | | |
| 146 | Ran-specific GTPase-activating protein | sp\|P43487\|RANG_HUMAN | RANBP1 | 23 kDa | 2 |
| | OS = Homo sapiens | | | | |
| | OX = 9606 GN = RANBP1 PE = 1 SV = 1 | | | | |
| 147 | Protein disulfide-isomerase A3 OS = Homo sapiens | sp\|P30101\|PDIA3_HUMAN | PDIA3 | 57 kDa | 2 |
| | OX = 9606 GN = PDIA3 PE = 1 SV = 4 | | | | |
| 148 | Annexin A7 OS = Homo sapiens | sp\|P20073\|ANXA7_HUMAN | ANXA7 | 53 kDa | 2 |
| | OX = 9606 GN = ANXA7 PE = 1 SV = 3 | | | | |
| 149 | Lysosome-associated membrane glycoprotein 1 | sp\|P11279\|LAMP1_HUMAN | LAMP1 | 45 kDa | 2 |
| | OS = Homo sapiens OX = 9606 | | | | |
| | GN = LAMP1 PE = 1 SV = 3 | | | | |
| 150 | Ras-related C3 botulinum toxin substrate 1 | sp\|P63000\|RAC1_HUMAN | RAC1 | 21 kDa | 2 |
| | OS = Homo sapiens | | | | |
| | OX = 9606 GN = RAC1 PE = 1 SV = 1 | | | | |

Importantly, Bla and VSV-G were also detected, indicating our EVs incorporate Bla-tetraspanins and viral Env fusion proteins as expected. Interestingly, a t-SNARE (STX4) and one of its accessory proteins (STXBP3) were also detected in the EV sample. To further characterize the purified EVs, morphological analysis of both VSV-G- and non-enveloped-EVs was performed using transmission electron microscopy. The EM micrographs revealed highly similar, spherical, 50-150 nm diameter membrane-enclosed particles for both conditions (FIG. 2e). These analytical characterizations reveal that the purified vesicles have a size and protein content consistent with EVs, primarily small or medium EVs (s/m-EVs), and also incorporate the Bla and VSV-G proteins.

HEK-EVs show no evidence of spontaneous membrane fusion, yet become highly fusogenic when incorporating VSV-G fusion protein

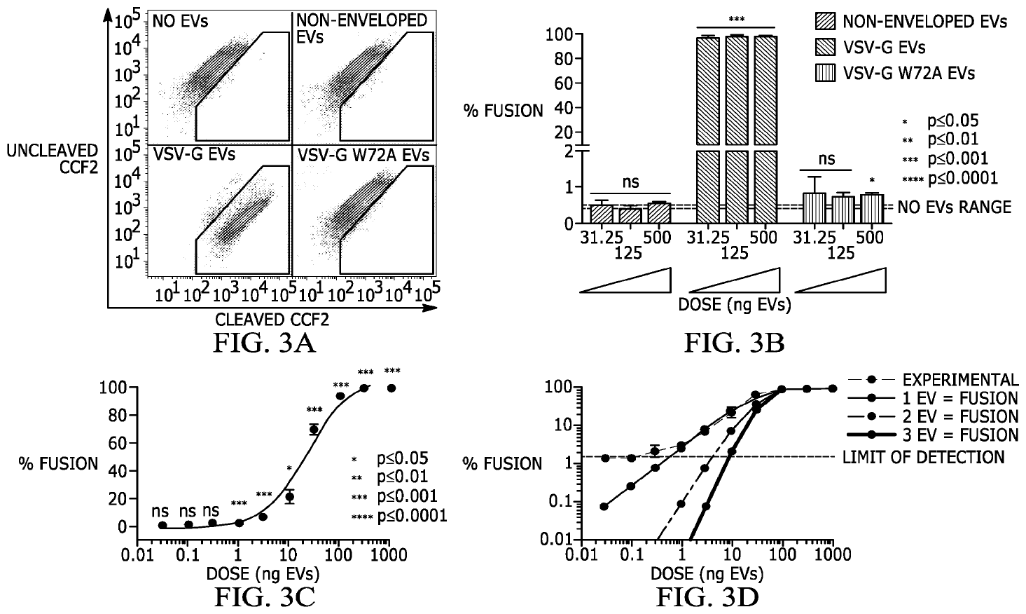

Next, we tested the ability of HEK-EVs incorporating the Bla-tetraspanins to fuse with target Jurkat E6 cells over a range of EV concentrations. In order to determine the background of our assay, we utilized a negative control in which only PBS was added to target cells. Consistent with the previous use of this assay to measure viral fusion, a very small amount of dye conversion was observed in this negative control (FIG. 3a, No EVs panel). We then added EVs containing Bla-tetraspanins to target cells to determine if they are capable of fusion. Surprisingly, EVs showed fusion levels no higher than PBS controls at all 3 concentrations tested (FIGS. 3a-b, Non-Enveloped EVs condition). This indicates that there is no evidence of spontaneous EV fusion that is detected with our assay. As a positive control to demonstrate that the Bla-tetraspanin constructs were functional, we harvested EVs from cells co-transfected with the VSV-G Env that confers fusogenicity to lentiviral particles. Interestingly, VSV-G EVs were highly fusogenic compared to the PBS control, even at the lowest EV dose tested (FIGS. 3-b, VSV-G EVs condition). These data indicate that our Bla-tetraspanin constructs are indeed functional and lend additional support to our finding that non-enveloped do not spontaneously fuse. Furthermore, it suggests that viral envelope proteins are capable of conferring high-level fusogenicity to EVs. To demonstrate that the fusogenicity of VSV-G EVs was related to the fusion machinery of VSV-G, we made Bla-EVs incorporating the VSV-G W72A mutant. A t all doses tested, VSV-G W72A EVs lost nearly all of their fusogenicity compared to their WT counterparts (FIGS. 3a-b, VSV-G W72A EVs condition). Taken together, we find no evidence that EVs are spontaneously fusogenic, but expression of viral envelope glycoproteins with intact fusion machinery can impart high levels of fusogenicity.

VSV-G HEK-EVs are highly fusogenic in a dose-dependent manner

To further investigate the fusogenicity of VSV-G HEK-EVs, we performed a dose-titration curve using Jurkat target cells. As expected, we observed a dose-dependent response with VSV-G EVs showing fusion levels significantly higher than PBS negative controls even at doses as low as ing EVs (FIG. 3c). Dose response curves of EV fusion can also be used to calculate the number of EVs required to generate a fusion signal. Briefly, Poisson distribution can be used to model the probability of a fusion event occurring based on the ratio of EVs to cells, and distinct decay curves are predicted as the EV dose is lowered if 1, 2, or 3 EVs are required to generate a fusion signal. A more detailed explanation of Poisson distribution and these calculations can be found in the STAR methods section. Using our VSV-G EV dose curve data, we set the ratio of EVs to cells to match the fusion observed in our 10Ong EV condition (-93% fusion), and modeled the Poisson distribution predicted curves based on fusion thresholds of 1, 2, and 3 EVs (FIG. 3d). The experimental VSV-G EV fusion data in FIG. 3c was extremely similar to the Poisson distribution curve predicted by a fusion threshold of 1 EV, down to the limit of detection of our assay (-1% fusion), and was distinct from the curves predicted if 2 or 3 EVs were required for a fusion signal. Together, these data suggest that VSV-G EVs are potently fusogenic in a dose-dependent manner and that our assay is capable of detecting even a single EV fusing with a cell. This remarkable level of sensitivity further supports our previous finding that non-enveloped EVs are not spontaneously fusing with cells.

EVs from multiple producer cells show no evidence of inherent fusion activity

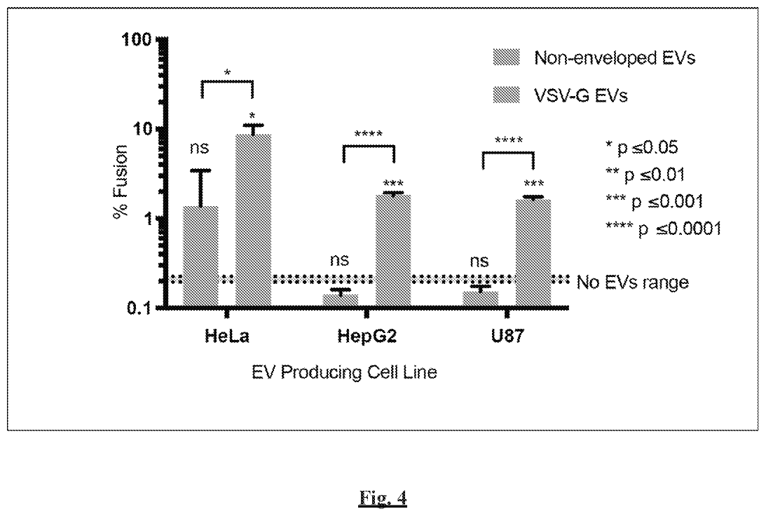

EVs are known to incorporate proteins representative of those highly expressed in the producer cells. We reasoned that HEK293Ts might not express proteins that could enable HEK-EV fusion and that these proteins might be present in other cell lines. To test whether other cell lines produce naturally fusogenic EVs, we individually transfected HeLa, HepG2, and U87 cells with the Bla-tetraspanin constructs, with or without the addition of VSV-G. Non-enveloped EVs harvested from these cells showed no evidence of fusion, supporting our previous data suggesting that EVs by themselves do not appear to be capable of full fusion and content mixing (FIG. 4). In marked contrast, VSV-G EVs from these 3 cell lines were all significantly fusogenic compared to negative controls, although not as fusogenic as VSV-G HEK-EVs. Taken together, these data reinforce our findings that EVs are not inherently fusogenic, regardless of producer cell choice, but become fusogenic upon expression of an exogenous viral fusion protein.

The HERV-W Env protein Syncytin-1 confers fusogenic activity to EVs

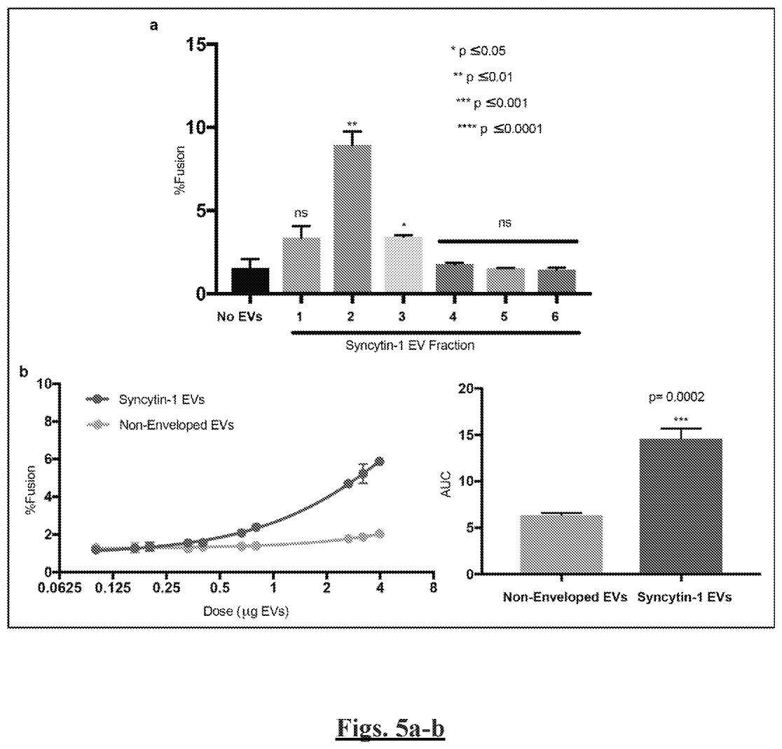

To investigate whether Syncytin-1 could confer fusogenicity to non-fusogenic EVs, we transfected HEK293Ts with Bla-tetraspanins with or without Syncytin-1 fusion protein. Using our fusion assay, we tested 6 different eluent fractions for fusion into HEK293T target cells. Indeed, we found that Syncytin-1 EVs were significantly fusogenic when compared to PBS negative controls (p=0.0012, fraction 2, FIG. 5a). Finally, the fusion of Syncytin-1 EVs at different doses was compared to that of non-enveloped EVs. Syncytin-1 EVs were significantly more fusogenic than non-enveloped EVs in a dose-dependent manner (p=0.0002, FIGS. 5b-c), although fusion was considerably lower than observed with VSV-G EVs. These findings demonstrate that Syncytin-1 can confer significant fusogenicity to EVs, providing a mechanism whereby EVs can acquire fusion capability in the absence of an exogenous viral infection and provide a mechanism for EV-cell fusion in cancer.

EVs from the breast cancer cell line MCF-7 are naturally fusogenic in a manner regulated by Syncytin-1 and its cognate receptor SLC1A5

Considering our finding that Syncytin-1 can mediate EV fusion, we obtained two breast cancer cell lines reported to express Syncytin-1: MCF-7 and MDA-MB-231. To investigate if either of these cell lines could produce naturally fusogenic EVs, we transfected them with the Bla-tetraspanin constructs and harvested EVs. EVs from these breast cancer cells were used in the CCF2-AM fusion assay, with each cell line also serving as a target cell. Provocatively, MCF-7 cells produced EVs naturally capable of fusing with MDA-MB-231 cells (p=0.0032, FIG. 5d), although fusion levels were low overall. In order to probe whether fusion was due to Syncytin-1 on the EV surface, we overexpressed its receptor, SLC1A5, on the MDA-MB-231 target cells. We hypothesized that Syncytin-1 mediated EV fusion would increase under conditions of SLC1A5 overexpression. Indeed, the addition of exogenous SLC1A5 to MDA-MB-231s increased the fusion of MCF-7 EVs, implicating Syncytin-1 as a causative EV fusion mediator for some cancers (p=0.0006, FIG. 5e).

SLC1A5-containing EVs fuse with Syncytin-1 expressing cancer cells

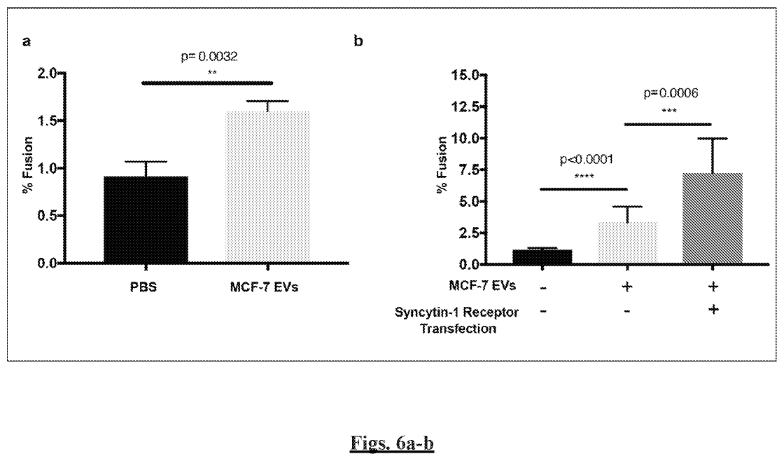

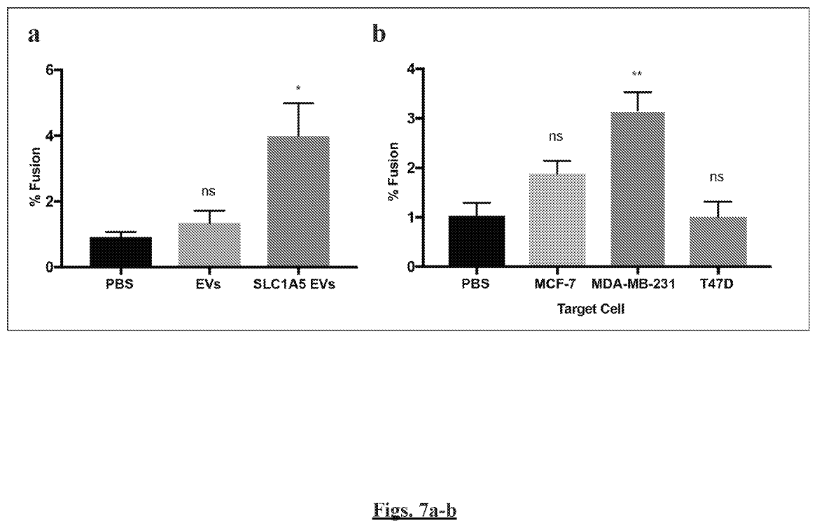

EVs were produced in HEK293Ts co-transfected with SLC1A5 and the Bla-tetraspanin plasmids. Mass spectrometry confirmed elevated levels of EV-associated SLC1A5 upon overexpression (data not shown). Continuing with the use of our MDA-MB-231 breast cancer cells as targets due to their Syncytin-1 expression, we used them in the fusion assay with SLC1A5 EVs. Interestingly, SLC1A5 EVs fused with MDA-MB-231 cells while identical EVs lacking overexpressed SLC1A5 did not (p=0.0128, FIG. 6a). The finding that SLC1A5 allowed EVs to fuse with MDA-MB-231 cells led us to probe fusion into other breast cancer cell lines to determine if SLC1A5 EVs could be used therapeutically to target Syncytin-1 expressing cancer cells. We observed elevated fusion of SLC1A5 EVs with MCF-7 cells although these data did not reach statistical significance (p=0.1056, FIG. 6b). SLC1A5 EVs did not fuse with T47D breast cancer cells, which have not been shown to express Syncytin-1 (p=1.00). Finally, SLC1A5 EVs again demonstrated fusion with MDA-MB-231 cells in this experiment (p=0.0018) (FIGS. 7a-b). The finding that EVs from HEK293T cells without SLC1A5 overexpression were not fusogenic despite having some SLC1A5 detectable by MS indicates that the density of the receptor may be critical to effective fusion with MDA-MB-231s. Together, these data implicate SLC1A5, in addition to its cognate Syncytin-1, in playing a role in EV fusion and communication in the context of cancer. Additionally, these data demonstrate the feasibility of using SLC1A5 to engineer therapeutic EVs that preferentially fuse with Syncytin-1 expressing cancer cells (FIG. 8).

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

The following is claimed:

1. An isolated, fusogenic particle comprising:
a lipid envelope associated with at least one targeting protein, the at least one targeting protein being a viral fusion protein or a cognate receptor of a viral fusion protein; and
a therapeutic agent contained within the fusogenic particle.

2. The fusogenic particle of claim 1, wherein the lipid envelope is a mono- or bi-layer lipid structure.

3. The fusogenic particle of claim 2, wherein the lipid envelope is a mono- or bi-layer lipid structure is an extracellular vesicle (EV).

4. The fusogenic particle of claim 2, wherein the lipid envelope is selected from an exosome, a microsome, an endosome, an enveloped virus, an enveloped viral-like particle, a nanosome or a vacuole.

5. The fusogenic particle of claim 1, wherein the viral fusion protein is derived from a population of circulating exogenous viral fusion proteins.

6. The fusogenic particle of claim 5, wherein the viral fusion protein is a viral envelope glycoprotein.

7. The fusogenic particle of claim 6, wherein the viral envelope glycoprotein is vesicular stomatitis virus glycoprotein (VSV-G).

8. The fusogenic particle of claim 1, wherein the viral fusion protein is derived from a population of endogenous viral fusion proteins.

9. The fusogenic particle of claim 8, wherein the viral fusion protein is derived from a human retrovirus.

10. The fusogenic particle of claim 9, wherein the viral fusion protein is Syncytin-1.

11. The fusogenic particle of claim 1, wherein the cognate receptor is solute carrier family 1 member 5 (SLC1A5).

12. The fusogenic particle of claim 1, being formulated as a pharmaceutical composition.

13. A method of delivering a therapeutic agent to a cell, the method comprising contacting the cell with an effective amount of the fusogenic particle of claim 1.

14. The method of claim 13, wherein the lipid envelope is a mono- or bi-layer lipid structure.

15. The method of claim 14, wherein the lipid envelope is a mono- or bi-layer lipid structure is an EV.

16. The method of claim 14, wherein the lipid envelope is selected from an exosome, a microsome, an endosome, an enveloped virus, an enveloped viral-like particle, a nanosome or a vacuole.

17. The method of claim 13, wherein the viral fusion protein is derived from a population of circulating exogenous viral fusion proteins.

18. The method of claim 17, wherein the viral fusion protein is a viral envelope glycoprotein.

19. The method of claim 18, wherein the viral envelope glycoprotein is VSV-G.

20. The method of claim 13, wherein the viral fusion protein is derived from a population of endogenous viral fusion proteins.

21. The method of claim 20, wherein the viral fusion protein is derived from a human retrovirus.

22. The method of claim 21, wherein the viral fusion protein is Syncytin-1.

23. The method of claim 13, wherein the cognate receptor is SLC1A5.

24. The method of claim 13, wherein the cell is in vitro.

25. The method of claim 13, wherein the cell is in vivo.

26. The method of claim 25, wherein the cell is a cancer cell.

27. The method of claim 26, wherein the cancer cell is located within a tumor or an organ.

28. The method of claim 26, wherein the cancer cell expresses endogenous retroviral glycoproteins and the fusogenic particle includes one or more cognate receptors that bind the retroviral glycoproteins.

29. The method of claim 25, wherein the cell is a virally-infected cell.
